(19) United States
(12) Patent Application Publication
(10) Pub. No.: US 2011/0076258 A1
(43) Pub. Date: Mar. 31, 2011

(54) METHODS OF MODULATING T CELL- DEPENDENT IMMUNE RESPONSES

(75) Inventors: Fabio Grassi, Bellinzona (CH); Ursula Schenk, Contra (CH); Camillo Ricordi, Miami, FL (US)

(73) Assignee: Converge Biotech Inc., Miami, FL (US)

(21) Appl. No.: 12/920,459

(22) PCT Filed: Mar. 3, 2009
(86) PCT No.: PCT/US09/01380

§ 371 Date: Nov. 22, 2010

Related U.S. Application Data
(60) Provisional application No. 61/068,034, filed on Mar. 3, 2008.
(60) Provisional application No. 61/200,944, filed on Dec. 5, 2008.

(51) Int. Cl. A61K 35/12; A61K 38/08; A61K 31/7076; A61P 19/02; A61P 37/06; A61P 3/10; A61P 25/00; C12N 5/00

(52) U.S. Cl. 424/93.71; 514/21.6; 514/47; 514/6.9; 514/17.9; 514/16.6; 435/375

(57) ABSTRACT

The present invention provides methods and compositions for modulating at least one T cell-dependent immune response using an inhibitor of ATP-mediated T cell activation, such as oxidized ATP, for therapeutic and research purposes.

### BACKGROUND

The T lymphocyte population comprises millions of cells, each of which expresses at the cell surface a unique receptor (TCR, T cell receptor). This heterogeneity allows the specific recognition of different pathogen-derived antigens by distinct T cells. The recognition of self-antigens is avoided through induction of self-tolerance. However, recognition of self-antigens in pathological situations leads to autoimmunity and self-destructive inflammatory disorders. T lymphocyte dependent inflammatory disorders include, for example, asthma, allergies, rheumatoid arthritis, psoriatic arthritis, arthritis, endotoxemia, type I diabetes, inflammatory bowel disease (IBD), colitis, multiple sclerosis, transplant rejection, graft-versus-host disease, amyotrophic lateral sclerosis, demyelinating disorders, scleroderma, Sjogren syndrome, Erdheim-Chester syndrome, Crohn's Disease syndrome, Takayasu arteritis, sarcoidosis, autoimmune hemolytic anemia, Werlhof's idiopathic thrombopenic syndrome, and dermatological conditions such as psoriasis, cutaneous T-cell lymphoma, cutaneous graft-versus-host disease, atopic dermatitis, allergic contact dermatitis, alopecia areata, vitiligo, drug-related eruptions, contact hypersensitivity, lupus erythematosus, pityriasis lichenoides et varioliformis, pityriasis lichenoides chronica, eczema, and lichen planus.

T cell activation depends on calcium signaling. Cytosolic calcium elevations represent an essential clue in the regulation of the adaptive immune response. Triggering of the B cell or T cell receptors leads to calcium (Ca²⁺) release from the ER and this in turn activates the opening of Ca²⁺ release associated (CRAG) channels in the plasma membrane leading to capacitative calcium entry (CCE) (Putney and Bird, Cell 75:199-201(1993)). The activation of the phosphatase calcineurin, which dephosphorylates the transcription factor NFAT and thus determines its nuclear translocation, constitutes an example of a Ca²⁺-dependent event crucial for successful T cell activation (Goldsmith and Weiss, Science 240:1029-1031(1988)).

The increase in cytosolic Ca²⁺ is paralleled by active mitochondrial Ca²⁺ uptake. Mitochondria serve as a high capacity Ca²⁺ sink, which helps to avoid cellular Ca²⁺ overload, and in addition contribute to a rapid clearing of Ca²⁺ in spatially restricted areas. The latter function of mitochondria critically modulates the activity of Ca²⁺-sensitive proteins. For example, mitochondrial Ca²⁺ buffering near the IP3 receptor on the ER was shown to increase the dynamic range of IP3 sufficient for CRAC activation (Gilabert et al., EMBO J 20:2672-2679 (2001)), whereas Ca²⁺ buffering near CRAC channels decreases the rate of their Ca²⁺ dependent inactivation (Hoth et al., Proc Natl Acad Sci USA 97:10607-10612; Hoth et al., J Cell Biol 137:633-648 (1997)). Finally, mitochondrial Ca²⁺ uptake stimulates, through the activation of the pyruvate, α-ketoglutarate and isocitrate dehydrogenases, the aerobic synthesis of adenosine triphosphate (ATP) (Jouaville et al., Proc Natl Acad Sci USA 96:13807-13812 (1999); Hajnoczky et al., Cell 82:415-424 (1995)). The Ca²⁺ dependent production of ATP covers the higher energy demand of stimulated cells, but may also modulate other ATP-regulated processes.

Indeed, ATP is also a ubiquitous extracellular messenger (Burnstock, Trends Pharmacol Sci 27:166-176 (2006)); Burnstock, Novartis Found Symp 276:26-48; discussion 48-57, 275-281 (2006)), which may be released into the extracellular space either by exocytosis of secretory vesicles or through gap junction hemichannels (Coco et al., JBC 278:1354-1362 (2003); Cotrina et al., PNAS 95:15735-15740 (1998); Bao et al., FEBS Letters 572:65-68 (2004)). ATP activates plasma membrane receptors for extracellular nucleotides termed P2 receptors, which are expressed in varying combinations by virtually all cells (Burnstock, Novartis Found Symp 276:26-48; discussion 48-57, 275-281 (2006)). P2 receptors are classified into two subgroups termed P2X and P2Y receptors. P2X 1-7 receptors all bind ATP and open to non-selective, often rapidly desensitizing ion channels. P2Y1, 2, 4, 6, 11-14 receptors preferentially bind ADP, UDP, UTP or UDP-glucose, they belong to the family of G-protein coupled receptors and their activation is linked to CCE.

There is increasing evidence that activation of P2 receptors on lymphocytes, in particular on T cells, crucially contributes to the outcome of TCR stimulation (Scrivens and Dickenson, Br J Pharmacol 146:435-444 (2005); Baricordi et al., Blood 87:682-690 (1996); Loomis et al,. J Biol Chem 278:4590-4596 (2003)). The half-life of ATP in the extracellular milieu is quite short since it is readily degraded to adenosine by the combined action of ectoapyrase (CD39) and ecto-5′-nucleotidase (CD73) (Yegutkin et al., Biochem J 367:121-128 (2002)). Interestingly, Treg cells have been shown to mediate T cell suppression through the action of these surface enzymatic activities (Deaglio et al., J Exp Med 204:1257-1265 (2007); Borsellino et al., Blood 110(4):1225-1232 (2007)). In spite of these emerging evidences for a regulatory role of ATP during the immune response, its source has not been clearly identified. ATP release from dying cells or damaged blood platelets as well as from mechanical stimulation has been proposed (Cotrina et al., PNAS 95:15735-15740 (1998); Bao et al., FEBS Letters 572:65-68 (2004)).

We have recently described an experimental model in which immunodeficient mice were reconstituted with hematopoietic progenitors from fetal liver of calreticulin (CRT) deficient embryos. CRT is a chaperone protein and the most important Ca²⁺ buffer in the ER (Ellgaard and Helenius, Nat Rev Mol Cell Biol 4:181-191 (2003)); its deletion in knock-out mice is lethal at day 12-13 of gestation (Mesaeli et al., J Cell Biol 144:857-868 (1999)). The amount of Ca²⁺ bound to CRT in the ER may dramatically influence the cell fate, e.g., overexpression of CRT results in increased susceptibility to apoptotic stimuli (Pinton et al., EMBO J 20:2690-2701 (2001)), whereas CRT-deficient cells are more resistant to apoptosis (Nakamura et al., J Cell Biol 150:731-740 (2000)). Crt^{-/-} T cells are hyper-responsive to antigenic stimulation and this perturbed T cell responsiveness determines in crt^{-/-} fetal liver chimeras (FLC) a severe immunopathological condition that mimics in some aspects the phenotype of graft-versus-host disease. This severe immunopathological condition is promoted by an oscillatory Ca²⁺ response to TCR triggering with prolonged activation of the MAPK pathway and protracted nuclear localization of NFAT1 (Porcellini et al., J Exp Med 203:461-471 (2006)).

We show here that crt^{-/-} T cells are characterized by increased mitochondrial Ca²⁺ buffering during CCE, leading to a higher rate of ATP synthesis. Release of ATP from T cells in the course of activation results in autocrine activation of P2X receptors with MAPK activation. This represents an essential costimulatory factor for productive T cell activation and expansion. We show that activity induced synthesis and release of ATP plays a crucial role in the outcome of T cell dependent inflammation and represents a possible pharmacological target for T cell immunosuppression.

Activation of P2X receptors is inhibited by ATP antagonists, such as oxidized ATP (oATP). Therefore, oATP is capable of exerting an anti-inflammatory effect by antagonizing the pro-inflammatory action of ATP on various cells of the immune system implicated in inflammation and tissue destruction, e.g., T cells.

We also show here that oATP in conjunction with anti-CD3 and syngeneic irradiated splenocytes induces skewing toward regulatory T cells (Treg cells) with high expression levels of Foxp3. Treg cells actively suppress effector T cell proliferation and cytokine production, and provide a mechanism of T cell tolerance separate from the development of a nonresponsive state in effector T cells (T cell anergy). The suppressive activity of Treg cells may be antigen-specific or antigen-nonspecific. In inflammatory bowel disease (IBD) induced by CD4⁺ naive cells with low numbers of Tregs, treatment with oATP significantly increased Foxp3 expression in Tregs and completely prevented immunopathology in the gut. Tregs have been shown to inhibit mast cell degranulation (Gri et al., Immunity 29:771-781 (2008)), and can thus play a role in suppressing immune responses involved in conditions associated with degranulation of mastocytes (e.g., asthma, allergy and anaphylactic shock). The importance of Treg cells in establishing and maintaining T cell tolerance has generated significant interest in methods for expanding Treg cells in vitro for therapeutic purposes, e.g., adoptive Treg cell therapy. Expanded Treg cell infusions can be used, e.g., to modulate the immune response; induce tolerance to cell, tissue and organ transplants; and treat autoimmune conditions. However, such clinical applications have been delayed by the challenge of successfully expanding Treg subpopulations without significant contamination from effector cells, e.g., Th17 cells, that may emerge from Foxp3⁺ selection. Such contaminating cells may outgrow Treg cells. Further, Treg cells may lose suppressive activity after repetitive stimulation in vitro. For these reasons, it is critical to begin Treg cell differentiation and/or expansion with an appropriate cell product and to selectively favor the generation and expansion of Treg cells over contaminating cells in vitro. We show here that treatment with oATP inhibited the conversion of Treg cells in vitro to Th17 lineage, as scored by RORγT expression.

As described above, we have discovered that ATP is released by activated T cells upon activation and the secreted ATP interacts with receptors on the T cell surface, acting as an autocrine as well as paracrine stimulus for protracted T cell activation. The blockade of this interaction by oATP leads to abortive T cell activation upon antigen encounter and generation of a state of T cell “unresponsiveness,” which ultimately results in reduced tissue destruction. Further, as described above, we have discovered that compositions comprising oATP can be used to induce both differentiation and significant expansion of immunosuppressive Treg cells, to maintain the Treg cell phenotype, and to enhance Treg cell immune suppressive activity, also resulting in reduced tissue destruction. Treatment with oATP could thus be beneficial before, during or after organ transplantation to avoid T cell-mediated rejection and in patients suffering from T cell-mediated graft-versus-host disease before,during or after bone marrow transplant.

Accordingly, it is an object of the present invention to treat immune or inflammatory conditions by contacting T cells with at least one agent that modulates a T cell-dependent immune response, such as an agent that inhibits ATP-mediated T cell activation and/or induces differentiation and expansion of Treg cells; e.g., oATP, the PX10 peptide or carbenoxolone. Such effects may have multiple therapeutic applications in, for example, treatments relating to immunological tolerance, autoimmunity, immunosuppression, and immunotherapy.

### SUMMARY OF THE INVENTION

The invention may be embodied in a method for modulating one or more T cell-dependent immune responses.

In one embodiment, the invention provides a method for inhibiting at least one T cell activity, comprising the step of contacting a T cell with an agent that inhibits ATP-mediated T cell activation. In some embodiments, the T cell activity is selected from the group consisting of activation, proliferation, differentiation, survival, cytolytic activity and cytokine production. In a preferred embodiment, the method for inhibiting at least one T cell activity is performed in vivo.

In one embodiment, said agent that inhibits ATP-mediated activation of T cells is a P2X receptor antagonist, e.g., a P2X7 receptor antagonist. In a preferred embodiment, said agent is oATP. In another embodiment, said agent is an agent that inhibits the permeability of pannexin hemichannels. In a preferred embodiment, said agent is the PX10 peptide (SEQ ID NO: 1) or carbenoxolone.

The invention also provides a method for inducing T cell anergy, comprising the step of contacting a T cell with an inhibitor of ATP-mediated T cell activation. In a preferred embodiment, the method for inducing T cell anergy is performed in vivo. In one embodiment, said agent that inhibits ATP-mediated activation of T cells is a P2X receptor antagonist, e.g., a T cell P2X7 receptor antagonist. In a preferred embodiment, said agent is oATP. In another embodiment, said agent is an agent that inhibits the permeability of pannexin hemichannels. In a preferred embodiment, said agent is the PX10 peptide (SEQ ID NO: 1) or carbenoxolone.

In one embodiment, the contacted T cell is an IL-17 secreting T cell (i.e., a TH17 cell).

In another embodiment, the invention provides a method for inducing the differentiation and/or expansion of Treg cells comprising the step of contacting a Treg cell with an agent that induces the differentiation and/or expansion of Treg cells. In a preferred embodiment, the method is performed in vitro. In an exemplary embodiment, the agent is a composition comprising oATP. The composition preferably also comprises (1) a T cell primary stimulator; and (2) a cellular component or a soluble mediator.

In another embodiment, the invention provides a method for inhibiting the conversion of Treg cells to non-Treg cells, comprising the step of contacting the Treg cells with an agent that inhibits the conversion of Treg cells to non-Treg cells. The non-Treg cells may be pathogenic T cells, e.g., Th17 cells. In a preferred embodiment, the agent is a composition comprising oATP. The method may be performed, e.g., in vitro or in vivo.

In another embodiment, the invention provides a method for converting non-Treg cells to Treg cells, comprising the step of contacting the non-Treg cells with an agent that enhances the conversion of non-Treg cells to Treg cells. The non-Treg cells may be naïve or pathogenic T cells, e.g., Th17 cells. In a preferred embodiment, the agent is a composition comprising oATP. The method may be performed, e.g., in vitro or in vivo.

In another embodiment, the invention provides a method for enhancing a Treg cell activity, e.g., an immune suppressive activity, by contacting the Treg cell with an agent that enhances a Treg cell activity. In a preferred embodiment, the agent is a composition comprising oATP. The method may be performed, e.g., in vitro or in vivo.

The invention may also be embodied in a method for treating cell death or tissue damage, comprising contacting a T cell with an agent that modulates one or more T cell-dependent immune responses. In one embodiment, the method may comprise one or more of the steps of: (1) contacting a T cell with an agent that inhibits ATP-mediated T cell activation; (2) contacting a Treg cell with an agent that inhibits its conversion to a non-Treg cell and/or enhances its immune suppressive activity; and (3) contacting a Treg cell with an agent that induces its differentiation and/or expansion. In a preferred embodiment, step (1) is performed in vivo. In a preferred embodiment, step (3) is performed in vitro and is followed by in vivo administration of the differentiated and/or expanded Treg cells. In a preferred embodiment, the agent or composition comprises oATP.

The invention may also be embodied in a method for treating an autoimmune or inflammatory condition, comprising the step of contacting a T cell with an agent that modulates at least one T cell-dependent immune response. In one embodiment, the method may comprise one or more of the steps of: (1) contacting a T cell with an agent that inhibits ATP-mediated T cell activation; (2) contacting a Treg cell with an agent that inhibits its conversion to a non-Treg cell and/or enhances its immune suppressive activity; and (3) contacting a Treg cell with an agent that induces its differentiation and/or expansion. In a preferred embodiment, step (1) is performed in vivo. In a preferred embodiment, step (3) is performed in vitro and is followed by in vivo administration of the differentiated and/or expanded Treg cells. In a preferred embodiment, the agent comprises oATP. In one embodiment, said autoimmune or inflammatory condition is an autoimmune or inflammatory condition of the adaptive immune system, e.g., a T lymphocyte-dependent inflammatory condition. In a preferred embodiment, said T lymphocyte-dependent inflammatory condition is associated with, for example, asthma, allergies, rheumatoid arthritis, psoriatic arthritis, arthritis, endotoxemia, type I diabetes, inflammatory bowel disease (IBD), colitis, multiple sclerosis, transplant rejection, graft-versus-host disease, amyotrophic lateral sclerosis, demyelinating disorders, scleroderma, Sjogren syndrome, Erdheim-Chester syndrome, Crohn's Disease syndrome, Takayasu arteritis, sarcoidosis, autoimmune hemolytic anemia, and Werlhof's idiopathic thrombopenic syndrome,. In another preferred embodiment, the T lymphocyte-dependent inflammatory condition is associated with a dermatological condition, such as, for example, psoriasis, cutaneous T-cell lymphoma, cutaneous graft-versus-host disease, atopic dermatitis, allergic contact dermatitis, alopecia areata, vitiligo, drug-related eruptions, contact hypersensitivity, lupus erythematosus, pityriasis lichenoides et varioliformis, pityriasis lichenoides chronica, eczema, and lichen planus.

In a preferred embodiment, the invention embodies a method of treating an immune or inflammatory condition in a subject in vivo. The method may comprise, e.g., administering to the subject in vivo at least one of (1) an agent that inhibits ATP-mediated T cell activation; (2) an agent that modulates at least one Treg cell activity, e.g., Treg cell differentiation and/or expansion or Treg cell immune suppressive activity; and (3) Treg cells differentiated and/or expanded by contact with an agent of the invention in vitro. In a preferred embodiment, the agent of step (1) and/or step (2) comprises oATP.

In one embodiment, the agent that modulates at least one T cell-dependent immune response (e.g., the agent that inhibits ATP-mediated T cell activation and/or the agent that modulates at least one Treg cell activity) is nanoencapsulated, e.g., to form nanoparticles. In another embodiment, Treg cells that have been differentiated and/or expanded by contact with an agent of the invention are nanoencapsulated, e.g., to form nanoparticles. In one embodiment, the nanoparticles are targeted to specific cells or tissues. In a preferred embodiment, the nanoparticles are targeted to specific cells or tissues in vivo.

The invention also provides methods of administering the agent that modulates a T cell-dependent immune response to a subject in need thereof. In one embodiment, the agent is administered intranodally. In another embodiment, the agent is administered topically. In another embodiment, the agent is administered intravenously or by injection.

The invention also provides methods of modulating a T cell-dependent immune response in a subject, such as an autoimmune disorder or an allergy, by administering Treg cells generated by induction of differentiation and/or expansion with an agent of the invention. The method comprises (a) obtaining a population of naïve T cells (e.g., naïve CD4⁺ T cells) from the subject; (b) producing Treg cells from the naïve T cells through differentiation and expansion; and (c) introducing the produced Treg cells into the subject to modulate, e.g., to suppress, the T cell-dependent immune response in the subject.

These and other objects and advantages of this invention will be more completely understood and appreciated by viewing the following more detailed description of the invention, taken in conjunction with the accompanying drawings.

### BRIEF DESCRIPTION OF THE DRAWINGS

FIG. 1: Increased mitochondrial Ca²⁺ buffering in crt^{-/-} T cells.
a) After ER Ca²⁺ depletion with tapsigargin, CCE was induced in crt^{-/-} and crt^{+/+} T cell clones by the addition of 0.5 mM Ca²⁺ to the extracellular medium (see Example 1). After complete washout of extracellular Ca²⁺, the mitochondrial Ca²⁺ buffering was visualized by the addition of ionomycin.
b) Histograms representing mitochondrial Ca²⁺ content after CCE with 0.5, 1 and 2 mM extracellular Ca²⁺ in crt^{-/-} and crt^{+/+} T cell clones.
c) Increased mitochondrial Ca²⁺ buffering in crt^{-/-} T cells retards CRAG inactivation. Cells were treated with tapsigargin followed by two separate additions of Ca²⁺ to the extracellular medium (see Example 1) (see also FIG. 2C). The rate of rise of cytosolic Ca²⁺ concentration was calculated at the first and second addition of Ca²⁺. A clear reduction was observed in crt^{+/+} but not in crt^{-/-} T cells.
d) When the same experiment was repeated in the presence of a mitochondrial uncoupler (CCCP), CRAG inactivation was comparable between ce^{+/+} and crt^{-/-} cells. (*, P<0.05, **, P<0.001, ***, P<0.0001)

FIG. 2: Reduced Ca²⁺ in the ER, unaltered mitochondrial membrane potential and decreased CRAC inactivation in crt^{-/-} T cells.
a) The ER Ca²⁺ content of Fura-2 loaded D011.10 TCR transgenic T cell clones was assessed by addition of the SERCA pump inhibitor tapsigargin to a medium devoid of Ca²⁺ (see Example 1). The passive leak of Ca²⁺ from the ER into the cytosol is seen as a slow transient rise in the cytosolic Ca²⁺ concentration. Crt^{-/-} T cells have a reduced ER Ca²⁺ content, calculated as area under the curve, compared to crt^{+/+} cells.
b) TMRM staining of sorted naïve and effector/memory (CD44⁺CD62L⁻) CD4 cells isolated from crt^{-/-} and crt^{+/+} FLC shows that the CRT deletion does not modify the mitochondrial membrane potential.
c) To score CRAC inactivation, T cells were loaded with Fura-2 and plated on poly-L-lysine coated coverslips (see Example 1). The ER calcium stores were depleted by the addition of tapsigargin in a medium devoid of Ca²⁺. Following complete ER store depletion Ca²⁺ was added twice for 100 s. Both Ca²⁺ additions result in Ca²⁺ influx through CRAC. Since these channels are inactivated by Ca²⁺ the second Ca²⁺ rise has a slower rate and amplitude. Crt^{-/-} cells showed diminished CRAC inactivation.

FIG. 3: Mitochondrial Ca²⁺ uptake during T cell activation leads to ATP synthesis and release.
a) ATP synthesis at different time points after T cell activation with CD3 antibodies in crt^{-/-} and crt^{+/+} sorted naïve CD4⁺ T cells (see Example 2).
b) ATP production upon T cell activation in the absence (control) or presence of the ATP synthetase inhibitor oligomycin.
c) FACS profile of CD62L expression on the surface of sorted naïve CD4 crt^{-/-} and crt^{+/+} T cells at 38 h following activation with plate bound CD3 and CD28 antibodies. The same experiment was performed in the presence of oATP.
d) Naïve CD4⁺ T cells were stained with the nucleotide binding compound quinacrine (see Example 2). The homogenous cytosolic staining indicates the absence of secretory vesicles containing ATP.
e) Subcellular fractionation of non-stimulated and activated naïve T cells on a continuous sucrose gradient (see Example 2). ATP was detected only in fractions containing also the cytosolic protein Zap-70 and not in those characterized by the small vesicular marker cellubrevin.

FIG. 4: Transcription of P2 receptors and Ca²⁺ responses by selective agonists in DO11.10 TCR transgenic T cell clones.
a) RT-PCR for P2 receptors shows that P2X1,4,7 are co-expressed together with P2Y1,12,13,14 in T cell clones (see Example 3).
b) Ca²⁺ imaging experiments in normal medium (first panel) or medium devoid of Ca²⁺ (second panel) confirm the presence of both ionotropic and metabotrobic P2 receptors on CD4 T cell clones.
c) Preferential agonists for P2X receptors (αβMeATP: P2X1, MeSATP: all P2X, BzATP: P2X7) more specifically confirm the functional competence of the receptors.
d) Functional P2Y receptors are present on T cell clones, as shown by the response to preferential agonists (MeSADP: P2Y1,12,13 and UDP-glucose: P2Y14).

FIG. 5: Role of pericellular ATP in protracted MAPK activation following TCR stimulation.
a) OVA specific crt^{-/-} T cell clones were stimulated with biotinylated CD3 antibodies followed by cross-linking with avidin (see Example 3). After 30 min of stimulation, cells were either left untreated (first panel on the left), or treated with the src-like kinase inhibitor PP2 either alone (second panel) or in combination with oATP (third panel) or ARL (fourth panel). PP2 efficiently inhibited TCR signaling, as shown by the dephosporylation of Zap-70, whereas protracted Erk activation was less affected. The combination of PP2 with oATP almost completely abolished Erk phosphorylation, whereas the combination of PP2 and the ectonuclease inhibitor ARL increased Erk activation at late time points.
b) Crt^{-/-} T cell clones were stimulated with cross-linked CD3 antibodies for 16 h (see Example 3). The first panels on the left show the characteristic protracted phosphorylation of p38 MAPK and Erk. These protracted activations were abolished by oligomycin as an inhibitor of mitochondrial ATP synthesis as well as by the two P2 receptor antagonists oATP and PPADS.

FIG. 6: Pharmacological inhibition of P2 receptors impairs T cell proliferation as well as IL-2 secretion and implements anergy.
a) FACS profiles showing dilution of CFSE fluorescence in sorted naïve CD4⁺ cells stimulated with plate-bound CD3 and CD28 (see methods section) in the absence (upper panels) or presence (lower panels) of oATP (see Example 4). The same experiment in the presence of IL-2 (middle panels) or PMA (right panels) is displayed. The number of cells with detectable marker (proliferating cells) in timed acquisitions is indicated.
b) A representative experiment showing the IL-2 concentrations measured by ELISA in culture supernatants of naïve CD4⁺ cells stimulated with plate-bound CD3 and CD28 antibodies for 48 h, and in the presence of oATP or PPADS. Gray bars represent IL-2 concentrations obtained when PMA was added to the sample.
c) Quantification of Egr2 and Egr3 transcripts by real time RT-PCR at 2 and 16 h after stimulation of sorted naïve CD4⁺ cells with plate-bound CD3 and CD28 antibodies either in the absence or presence of oATP (see Example 4). *, P<0.05
d) Cytosolic Ca²⁺ profiles following CD3 stimulation of OVA-specific T cell clones pre-incubated for 16 h with ionomycin, CD3 and CD28 antibodies alone or in combination with oATP or oATP and PMA (see Example 4).

FIG. 7: Prevention of diabetes in INS-HA transgenic RAG-2^{-/-} mice by oATP treatment.
a) Blood glucose levels in RAG-2^{-/-} mice expressing HA under the control of rat insulin promoter at day 12 after adoptive transfer of TCR 6.5 anti-HA transgenic CD4⁺ T cells (see Example 5). Mice were either left untreated or injected daily from day 1 to 10 after transfer with two doses, intravenously and intraperitoneally, of PBS or oATP.
b) Histopathological examination of hematoxilin-eosin stained sections (100×) shows severe destructive insulitis in pancreas from PBS-treated mice, whereas no relevant pathological findings were observed in pancreas from oATP-treated animals.
c and d) Histograms with numbers of transgenic TCR6.5⁺ cells recovered from the spleen c) and pancreas d) of PBS-and oATP-treated animals. Histograms on the right represent the percentage of CD69⁺ TCR 6.5⁺ cells recovered from the pancreas of the indicated mice. n.d., non detectable.
e) Splenocytes from PBS- and oATP-treated mice were stimulated in vitro with HA peptide and the indicated cytokines measured by cytometric beads array (see Example 5). Histograms display TNF-α, IFN-γ and IL-6 concentrations in the supernatants expressed as pg/ml per 10⁴ TCR6.5⁺ cells. (mean±SD, n=5) *, P<0.05, **, P<0.001, ***, P<0.0001.

FIG. 8: Amelioration of inflammatory bowel disease by oATP treatment.
a) Photographs of representative mesenteric lymph nodes, spleens and colons from cd3ε^{-/-} mice adoptively transferred with CD4⁺/CD25⁺ and CD4⁺ cells (see Example 5). The lower panel shows organs from mice reconstituted with CD4 cells and treated with oATP. Bar=1 cm
b) Histograms representing inflammation scores in animals treated as indicated (see methods section) (mean±SD; n=5, CD4⁺/25⁺ healthy control group; n=7, CD4⁺ untreated group; n=8, CD4⁺ oATP-treated group).
c) Hematoxilin/eosin (upper panels) and Alcian/PAS (lower panels) staining of colon sections from the indicated animals (all the microscopic pictures in the box were taken at the same magnification; scale bar=50 μm). In mice reconstituted with CD4⁺/CD25⁺ cells, no inflammatory changes are evident and a large number of goblet cells with voluminous Alcian-PAS-positive droplets lines the colonic crypts (arrowheads); in mice adoptively transferred with CD4⁺ cells and injected with oATP, the lamina propria is focally expanded by inflammatory cells infiltrate (arrow) and the colonic crypts epithelium shows moderate hyperplasia. Partial goblet cell depletion and reduction in size of Alcian-PAS-positive droplets are also noticeable (arrowheads); in mice reconstituted with CD4 cells and treated with PBS, the lamina propria is markedly expanded by inflammatory cell infiltrate with focal findings of crypt abscessation (arrow). Colonic crypts are also severely dysplastic with almost complete loss of goblet cells.
d) Cell recoveries from mesenteric lymph nodes and spleen of the indicated animals.

e) Absolute numbers of IL-2-, TNFα-, IFNγ- and IL-17-producing CD4⁺ T cells in mesenteric lymph nodes from the indicated mice (mean±SD; n=5, CD4⁺/25⁺ healthy group; n=7, CD4⁺ untreated group; n=8, CD4⁺ oATP-treated group). *, P<0.05, **, P<0.001, ***, P<0.0001
f) Absolute numbers of CD44⁺CD62L⁻ effector/memory and CD69⁺ CD4 cells recovered from mesenteric lymph nodes and spleen of the indicated group of animals (bars represent mean values).

FIG. 9: Inhibition of pannexin hemichannel assembly inhibits T cell activation and proliferation.
a) CFSE-loaded human T cells were stimulated with plate-bound anti-CD3/28 antibodies and proliferation was measured by FACS analysis (see Example 6). Note that the pannexin blocking peptide PX10 inhibited T cell proliferation comparably to oATP.
b) IL-2 secretion from CD3⁺/28⁺ stimulated mouse T cells into medium was strongly inhibited by both PX10 and oATP.

FIG. 10: The pannexin blocking peptide PX10 increases the intracellular ATP concentration upon TCR triggering.

The presence of the pannexin blocking peptide PX10 during TCR triggering increases the intracellular ATP concentration (see Example 6), suggesting that pannexin hemichannels represent an important route for ATP secretion in the course of T cell activation.

FIG. 11: oATP induces Treg cell differentiation and expansion.
a) Stimulation of naïve CD4⁺ T cells in the presence of oATP significantly enhanced the percentage of CD4⁺CD25^{high}Foxp3⁺ Treg cells.
b) Stimulation of sorted CD4⁺CD25^{high} cells comprising natural Treg cells in the presence of oATP induced the expansion of Treg cells with higher expression levels of Foxp3.
c) Analysis of master transcription factors for Th1 (T-bet), Th17 (RORγT) and Treg (Foxp3) lineages by quantitative RT-PCR in the first 6 days after anti-CD3 stimulation revealed the progressive upregulation of Foxp3 in the presence of oATP, as opposed to the progressive upregulation of T-bet in untreated cultures.

FIG. 12: oATP treatment inhibits Th17 differentiation and promotes Foxp3 expression.
a) oATP gradually increased the expression of Foxp3 while suppressing the expression of RORγT in T cells stimulated by anti-CD3 under Th17 skewing conditions (TGFβ and IL-6 conditioned medium).
b) The absolute number of CD4⁺CD25^{high} cells expressing Foxp3 by FACS analysis was increased under Th17 skewing conditions in the presence of oATP.
c) De-differentiation of sorted CD4⁺CD25^{high} natural Treg cells to the Th17 lineage was prevented by oATP.

FIG. 13: Amelioration of inflammatory bowel disease by oATP treatment in animals into which an insufficient number of Treg cells has been adoptively transferred.
a) Daily treatment with 100 μl of 3 mM oATP administered intravenously increased Foxp3 expression in Treg cells in a mouse model of IBD, where a number of Treg cells insufficient to control inflammation were adoptively transferred into the animals.
b) oATP treated animals showed no signs of bowel inflammation as well as no increase in spleen and mesenteric lymph node size.
c) oATP treated animals displayed reduced counts of effector/memory T cells in mesenteric lymph nodes.
d) The ratio of Treg/EM cells in mesenteric lymph nodes was not significantly changed by oATP treatment.

FIG. 14: Experimental protocol for Treg generation in a mouse model of inflammatory bowel disease.
CD3ε^{-/-} mice were adoptively transferred with 2×10⁵ naïve CD4⁺ T cells; 2×10⁵ naïve CD4⁺ T cells with oATP; 2×10⁵ naïve CD4⁺ T cells with administration of oATP sixteen hours after the transfer; or 2×10⁵ naïve CD4⁺ T cells with 10⁵ natural Treg cells. oATP-treated animals received daily intravenous oATP administration on days 2-5, no oATP administration on days 6 and 7, and daily intravenous oATP administration on days 8-12. Mice were analyzed for CD4⁺ subpopulations on day 28 (See Example 12).

FIG. 15: Treg cell generation with oATP treatment in a mouse model of inflammatory bowel disease.
a) Colons, spleens and mesenteric lymph nodes of test animals were assessed for inflammation fourteen days after the last injection of oATP (see Example 12).
b) FACS analysis identifying the percentage of CD4⁺CD25^{high}Foxp3 regulatory T cells.
c) FACS analysis identifying effector memory T cells as CD4⁺CD44⁺CD62L⁻ or CD4⁺CD25⁺CD69⁺. The ratio of Treg cells to effector memory T cells is shown.

FIG. 16: Amelioration of proteinuria in NZB/NZW F₁ mice by oATP treatment.

Graph representing protein concentration in urine from NZB/NZW Fᵢ mice treated either with PBS (control) or oATP. 25-week -old female mice received either PBS or oATP intravenously (3 mM in 100 μl, five days treatment, 2 days break) for 6 weeks (see Example 14). Ten animals received PBS and ten animals received oATP.

FIG. 17: Amelioration of SLE in NZB/NZW F₁ mice by oATP treatment.

Twenty-five week-old NZB/NZW F₁ female mice were injected intravenously with either PBS or oATP (3 mM in 100 μl, five days treatment, 2 days break) for six weeks and examined for proteinuria (upper panel) and other indicated parameters (lower panels) (see Example 14). **: P<0.01; ***: P<0.001.

FIG. 18: Inhibition of T cell effector functions in systemic lupus erythematosus by oATP.

Effector/memory (CD44⁺CD62L⁻) CD4⁺ cells recovered from the spleen of 25 week-old NZB/NZW F₁ female mice treated either with PBS or oATP (upper panel); IFN-γ and IL-4 secretion by CD4⁺ effector/memory T cells stimulated with plate bound anti-CD3 and anti-CD28 antibodies for 48 h detected by ELISA (lower panels) (see Example 14). **: P<0.01; ***: P<0.001.

FIG. 19: Clinical score variations in collagen-induced rheumatoid arthritis model.

Graph representing the mean variation from initial disease severity clinical score assessed in collagen-induced RA mice receiving oATP (3 mM in 100 μl) or control PBS intravenously for 12 days, in a dosage schedule of five days treatment, 2 days break, and five days treatment, starting at day 0 (see Example 15).

FIG. 20: Diminution of collagen-specific antibodies by oATP treatment. Type II collagen ELISA performed on samples from oATP-treated and control mice in a collagen-induced rheumatoid arthritis model (see Example 15).

FIG. 21: Reconstitution of recombinase-deficient mice with crt^{-/-} and crt^{+/+} fetal liver progenitors.

Genotypes of E13 embryos from crt^{+/+} x crt^{-/-} breeding were determined, crt^{-/-} and crt^{+/+}0 embryos selected and fetal liver progenitors injected into recombinase-deficient mice (see, e.g., Porcellini et al., J. Exp. Med. 203:461-471 (2006)) (see Example 16).

FIG. 22: Graft-versus-host disease-like phenotype of calreticulin-deficient fetal liver chimeric mice.

Phenotype of crt^{+/+} fetal liver chimera (FLC) at week 12 and crt^{-/-} FLC at weeks 8, 10 and 12 after transfer of hematopoietic progenitors. Progressive worsening of alopecia, blepharitis, hunched posture and wasting syndrome are seen in crt^{-/-} FLC (see Example 16).

FIG. 23: Epidermal hyperplasia and abundant granulocytes in superficial derma with focal infiltration of epidermis in crt-deficient fetal liver chimeras.

Hematoxylin and eosin stained sections show severe dermal granulocytic inflammatory infiltrate in the skin of crt^{-/-} FLC, as opposed to crt^{+/+} FLC, where inflammatory cells are virtually absent (bar=50 μm in left panels; bar=10 μm right panels) (see Example 16).

FIG. 24: Amelioration of blepharitis in cre mice injected with oATP.

Phenotype of crt^{-/-} FLC before treatment and after daily intravenous treatment with PBS or 6 mM oATP (100 μl) for 2 weeks (see Example 16).

FIG. 25: Histological improvement of blepharitis in crt^{-/-} fetal liver chimeras injected with oATP for two weeks.

Histopathological evaluation of skin biopsies executed in a blinded fashion. The graphs show selected examples of dramatic results seen with oATP treatment (see Example 16).

### DETAILED DESCRIPTION OF THE INVENTION

Definitions and General Techniques

The present invention is generally directed to methods for modulating at least one T cell activity, having multiple therapeutic applications for diverse treatments relating for example to immunological tolerance, autoimmunity, immunosuppression, and immunotherapy.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics, and protein and nucleic acid chemistry and hybridization described herein are those well known and commonly used in the art.

The term “T cell” or “T lymphocyte” as used generically herein may refer to, for example, helper T cells (e.g., TH1, TH2, TH9 and TH17 cells), cytotoxic T cells, memory T cells, regulatory/suppressor T cells (Treg cells), natural killer T cells, γδ T cells, and/or autoaggressive T cells (e.g., TH40 cells), unless otherwise indicated by context. In certain embodiments, the term “T cell” refers specifically to a helper T cell. In certain embodiments, the term “T cell” refers more specifically to a TH17 cell (i.e., a T cell that secretes IL-17). In certain embodiments, the term “T cell” refers to a Treg cell.

The term “Treg cell” as used herein refers to a CD4⁺CD25⁺Foxp3⁺ regulatory T cell (i.e., a CD25^{+bright} T cell). Use of other T cells with regulatory capabilities (e.g., Tr1 cells and Th3 cells) in the methods of the invention is also contemplated.

“T cell activity” as used herein refers to one or more of the immunological processes of, e.g., T cell activation, proliferation, differentiation and survival, as well as associated effector immune functions including cytolytic activity (Tc cells) and cytokine production (Th cells). In one embodiment, the compositions and methods disclosed herein can be used to reduce helper T cell (Th) responses, e.g., Th17 cell responses. In another embodiment, the compositions and methods disclosed herein can be used to reduce cytotoxic T cell (Tc) responses. In another embodiment, the compositions and methods disclosed herein can be used to induce differentiation, expansion, and/or immune suppressive activity of regulatory T (Treg) cells. In some embodiments of the invention, at least one helper T cell activity is reduced by at least 30%, 40%, 50%, 60%, 70%, 80%, 90%, 95%, 96%, 97%, 98%, 99%, or 100%. In some embodiments of the invention, at least one Treg cell activity is increased by at least 5%, 10%, 15%, 20%, 30%, 40%, 50%, 60%, 70%, 80%, 90%, 100%, 200%, 300%, 400%, 500%, 600%, 700%, 800%, 900%, or 1000%. Foxp3 acts as a quantitative regulator of Treg suppressive function rather than a simple molecular switch and it inhibits effector functions in responder cells in a dose-dependent manner (Allan et al. Eur. J. Immunol. 38: 3282-3289 (2008)) Assays for detecting and/or monitoring the above activities are numerous and are well-known in the art, e.g., assays for immune cell proliferation, release of cytokines, expression of cell surface markers, cytotoxicity, etc.

The term “ATP-mediated T cell activation” refers to the binding of ATP to P2 receptors on T cells, resulting in T cell activation and/or expansion. The T cell activation is ATP-mediated if, for example, the T cell activation and/or expansion can be blocked by at least 30%, 40%, 50%, 60%, 70%, 80%, 90%, 95%, 96%, 97%, 98%, 99%, or 100% with an agent that specifically blocks binding of ATP to P2 receptors on T cells (e.g., oATP). ATP-mediated T cell activation may be assayed, e.g., by analyzing MAPK activation, ERK phosphorylation, and/or IL-2 expression in the ATP-stimulated T cell population (see, e.g., Examples 3 and 4). In certain embodiments, MAPK activation, ERK phosphorylation, and/or IL-2 expression are reduced by 10%, 20%, 30%, 40%, 50%, 60%, 70%, 80%, 90% or 100% in a T cell population upon treatment with an agent that inhibits ATP-mediated T cell activation. ATP-mediated T cell activation may also be assayed, e.g., by monitoring ATP-mediated opening of ion channels through, for example, electrophysiological assays to measure the efflux of potassium out of the T cells or the influx of sodium or calcium into the T cells. In one embodiment, ATP is released from T cells in the course of activation, and results in autocrine and/or paracrine activation of P2 receptors.

The term “ATP-mediated T cell activation” is understood to include the binding of ATP to P2X receptors on T cells, resulting in T cell activation and/or expansion. This may be referred to as “ATP-mediated T cell activation through P2X receptors.” The term “ATP-mediated T cell activation” is understood to include the binding of ATP to P2X7 receptors on T cells (i.e., “ATP-mediated T cell activation through P2X7 receptors on T cells”). The T cell activation through P2X receptors is ATP-mediated if, for example, the T cell activation and/or expansion can be blocked by at least 30%, 40%, 50%, 60%, 70%, 80%, 90%, 95%, 96%, 97%, 98%, 99%, or 100% with an agent that specifically blocks binding of ATP to P2X receptors on T cells (e.g., oATP). ATP-mediated T cell activation through P2X receptors may be assayed, e.g., by analyzing MAPK activation, ERK phosphorylation, and/or IL-2 expression in the ATP-stimulated T cell population (see, e.g., Examples 3 and 4). In certain embodiments, MAPK activation, ERK phosphorylation, and/or IL-2 expression are reduced by 10%, 20%, 30%, 40%, 50%, 60%, 70%, 80%, 90% or 100% in a T cell population upon treatment with an agent that inhibits ATP-mediated T cell activation through P2X receptors. ATP-mediated T cell activation through P2X receptors may also be assayed, e.g., by monitoring ATP-mediated opening of ion channels through, for example, electrophysiological assays to measure the efflux of potassium out of the T cells or the influx of sodium or calcium into the T cells. As understood by the skilled worker, analogous assays may be used to monitor activation of other P2 receptor subtypes, e.g., P2Y receptors. In one embodiment, ATP is released from T cells in the course of activation, and results in autocrine and/or paracrine activation of P2X receptors.

The term “T cell anergy” refers to a state of reduced T cell activity, e.g., a state of T cell non-reactivity upon contact with an antigen. T cell anergy may result from, e.g., lack of co-stimulation and concomitant inadequacy of IL-2 production, preventing proliferation of the T cell.

The term “differentiation” as used herein in reference to T cells refers to a process by which a less specialized cell type becomes a more specialized cell type.

The term “expansion” as used herein in reference to T cells refers to an increase in the number of T cells.

The terms “inhibit” or “inhibition of” as used herein means to reduce by a measurable amount. Inhibition may be partial or complete.

The term “induce” or “induction of” as used herein means to increase by a measurable amount.

The term “immune condition” will be understood by those skilled in the art to include any condition that has an immune component associated with it, and/or any condition characterized by an immune or autoimmune response. The term “adaptive immune condition” will be understood by those skilled in the art to include any condition that has an adaptive immune system component associated with it.

The term “inflammation” will be understood by those skilled in the art to include any condition characterized by a localized or a systemic protective response, which may be elicited by physical trauma, infection, chronic diseases, such as those mentioned above, and/or chemical and/or physiological reactions to external stimuli (e.g., as part of an allergic response). Any such response, which may serve to destroy, dilute or sequester both the injurious agent and the injured tissue, may be manifested by, for example, heat, swelling, pain, redness, dilation of blood vessels and/or increased blood flow, invasion of the affected area by white blood cells, loss of function and/or any other symptoms known to be associated with inflammatory conditions.

The term “inflammation” will thus also be understood to include any inflammatory disease, disorder or condition per se, any condition that has an inflammatory component associated with it, and/or any condition characterized by inflammation as a symptom, including, inter alia, acute, chronic, ulcerative, specific, allergic and necrotic inflammation, and other forms of inflammation known to those skilled in the art. The term thus also includes, for the purposes of this invention, inflammatory pain and/or fever caused by inflammation.

The term “P2 receptor” refers to a type of receptor for extracellular nucleotides that includes, e.g., P2X and P2Y receptors. The term “P2X receptor” refers to an ATP-gated cation channel present on a variety of cell types. P2X 1-7 receptors all bind ATP and open non-selective, often rapidly desensitizing ion channels. The P2X7 receptor, for example, is largely present on cell types involved in the inflammatory/immune process; specifically, macrophages, mast cells and lymphocytes (T and B). Activation of the P2X7 receptor by extracellular nucleotides, in particular adenosine triphosphate (ATP), is known to lead, amongst other things, to the maturation and release of interleukin-1β (IL-1β).

As used herein, the term “P2X receptor antagonist” is a compound or other substance that is capable of preventing, whether fully or partially, activation of the P2X receptors (P2X1-7), as measured by any suitable assay such as those described and referenced below. A P2X receptor antagonist, may be, for example, an agent that competes with ATP for binding to a P2X receptor, e.g., oATP. A P2X7 receptor antagonist may be, for example, an agent that competes with ATP for binding to a P2X7 receptor. A T cell P2X7 receptor antagonist may be, for example, an agent that competes with ATP for binding to P2X7 receptors on T cells.

Methods for assaying P2X receptor antagonism are known in the art. For example, U.S. Pat. No. 6,720,452 describes an assay based on the observation that when the P2X7 receptor is activated using a receptor agonist in the presence of ethidium bromide (a fluorescent DNA probe), an increase in the fluorescence of intracellular DNA-bound ethidium bromide is observed. Thus, an increase in fluorescence can be used as a measure of P2X7 receptor activation and therefore to quantify the inhibitory effect of a compound or substance on the P2X7 receptor.

Examples of P2X7 receptor antagonists include, but are not limited to, the compounds described in U.S. Pat. Nos. 6,492,355; 6,720,452; 6,881,754 and 7,129,246, the entire contents of which are incorporated herein by reference.

As used herein, the term “pannexin” refers to a hemichannel forming protein that is homologous to the invertebrate innexin family of proteins. Pannexin hemichannels are known to allow passage of ATP in erythrocytes and taste receptor cells and determine IL1-β secretion in macrophages. Unless specifically indicated, “pannexin” may refer to either pannexin-1 or pannexin-2. In certain embodiments, the term “pannexin” refers to pannexin-1.

As used herein, the term “oATP” refers to oxidized ATP, which may be derived from ATP by oxidation of the hydroxyls present at the ribose 2′ and 3′ positions to dialdehydes. This oxidation can be carried out with a periodic acid salt, as described in P. N. Lowe et al., “Preparation and chemical properties of periodate-oxidized adenosine triphosphate and some related compounds”, Biochemical Society Transactions 7:1131-1133 (1979). oATP is thought to act as a P2z/P2X7 purinoceptor antagonist (Ferrari et al., J Exp Med 185(3):579-582 (1997)).

As used herein, the term “pannexin inhibitor” refers to an agent that inhibits the function of pannexin hemichannels. A pannexin inhibitor may be, e.g., a small molecule, an antibody, a peptide, or any other substance that interferes with pannexin hemichannel function. In one embodiment, the pannexin inhibitor is a peptide. In a preferred embodiment, the pannexin inhibitor is the PX10 peptide, or a peptide at least about 60%, 70%, 80%, 90%, 95%, 96%, 97%, 98%, or 99%, identical to the PX10 peptide for which the sequence is WRQAAFVDSY (SEQ ID NO: 1), wherein the N- and C-termini are, in certain embodiments, free and not modified.

As used herein, the twenty conventional amino acids and their abbreviations follow conventional usage. See Immunology—A Synthesis (2^{nd} Edition, E. S. Golub and D. R. Gren, Eds., Sinauer Associates, Sunderland, Mass. (1991)), which is incorporated herein by reference. Stereoisomers (e.g., D-amino acids) of the twenty conventional amino acids, unnatural amino acids such as α-, α-disubstituted amino acids, N-alkyl amino acids, lactic acid, and other unconventional amino acids may also be suitable components for peptides of the present invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, α-N-methylarginine, and other similar amino acids and imino acids (e.g., 4-hydroxyproline). In the peptide notation used herein, the lefthand direction is the amino terminal direction and the righthand direction is the carboxy-terminal direction, in accordance with standard usage and convention.

As applied to peptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity, and most preferably at least 99 percent sequence identity. Preferably, residue positions which are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, glutamic-aspartic, and asparagine-glutamine.

As discussed herein, minor variations in the amino acid sequences of peptides are contemplated as being encompassed by the present invention, providing that the variations in the amino acid sequence maintain at least 75%, more preferably at least 80%, 90%, or 95%, and most preferably 96%, 97%, 98%, or 99% of non-variant sequences. In particular, conservative amino acid replacements are contemplated. Conservative replacements are those that take place within a family of amino acids that are related in their side chains. Genetically encoded amino acids are generally divided into families: (1) acidic=aspartate, glutamate; (2) basic=lysine, arginine, histidine; (3) non-polar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the peptide derivative, e.g., inhibition of at least one T cell activity.

Preferred amino acid substitutions are those which: (1) reduce susceptibility to proteolysis, (2) reduce susceptibility to oxidation, (3) alter binding affinity for forming protein complexes, (4) alter binding affinities, and (5) confer or modify other physicochemical or functional properties of such analogs. Analogs can include various muteins of a sequence other than the naturally-occurring peptide sequence. For example, single or multiple amino acid substitutions (preferably conservative amino acid substitutions) may be made in the naturally-occurring sequence (preferably in the portion of the peptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized peptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et at. Nature 354:105 (1991), which are each incorporated herein by reference.

Peptide analogs are commonly used in the pharmaceutical industry as non-peptide drugs with properties analogous to those of the template peptide. These types of non-peptide compound are termed “peptide mimetics” or “peptidomimetics”. Fauchere, J Adv Drug Res 15:29 (1986); Veber and Freidinger, TINS p. 392 (1985); and Evans et al., J Med Chem 30:1229 (1987), which are incorporated herein by reference. Such compounds are often developed with the aid of computerized molecular modeling. Peptide mimetics that are structurally similar to therapeutically useful peptides may be used to produce an equivalent therapeutic or prophylactic effect. Generally, peptidomimetics are structurally similar to a paradigm peptide (i.e., a peptide that has a biochemical property or pharmacological activity), such as human antibody, but have one or more peptide linkages optionally replaced by a linkage selected from the group consisting of: —CH₂NH—, —CH₂S—, —CH₂—CH₂—, —CH═CH— (cis and trans), —COCH₂—, —CH(OH)CH₂—, and —CH₂SO—, by methods well known in the art. Systematic substitution of one or more amino acids of a consensus sequence with a D-amino acid of the same type (e.g., D-lysine in place of L-lysine) may be used to generate more stable peptides. In addition, constrained peptides comprising a consensus sequence or a substantially identical consensus sequence variation may be generated by methods known in the art (Rizo and Gierasch, Ann Rev Biochem 61:387 (1992), incorporated herein by reference); for example, by adding internal cysteine residues capable of forming intramolecular disulfide bridges which cyclize the peptide.

As used herein, the term “subject” refers to the recipient of a therapeutic treatment and includes all animals. In an exemplary embodiment, the subject is human.

As used herein, the terms “treat,” “treating” and “treatment” may be used to refer to decreasing, relieving or ameliorating a condition or disease, or at least one clinical symptom thereof. When administered before such symptom or condition is measurable, treatment may be considered “preventing.”

The term “agent,” as referred to herein, refers to a molecule, compound or composition. In some embodiments, an “agent” may comprise cells, e.g., Treg cells.

The term “therapeutic agent,” as referred to herein, refers to a molecule, compound, or composition that delivers a therapeutic effect.

The term “effective amount” refers to an amount of a therapeutic agent that confers a therapeutic effect on the treated patient when administered alone or in combination with another therapeutic agent. The effect may be objective (i.e. measurable by some test or marker) or subjective (i.e. the subject gives an indication of or feels an effect).

The term “agent that modulates a T cell-dependent immune response” refers to, e.g., inhibitors of ATP-mediated T cell activation and modulators of at least one Treg cell activity, e.g., T cell differentiation and/or expansion or T cell immune suppressive activity. In some embodiments, an “agent that modulates a T cell-dependent immune response” is a composition comprising Treg cells with immune suppressive activity.

### Inhibition of ATP-Mediated T Cell Activation

The present invention provides methods of inhibiting at least one helper T cell activity, e.g., T cell activation, proliferation, and/or effector function, using inhibitors of ATP-mediated T cell activation. In one embodiment, such inhibitors comprise agents that are P2X receptor antagonists, e.g.,T cell P2X7 receptor antagonists. In a preferred embodiment, the inhibitor is oATP. In another embodiment, such inhibitors comprise agents that inhibit pannexin hemichannel permeability. In another preferred embodiment, the inhibitor is the PX10 peptide (including analogs or chemically modified derivatives thereof) or carbenoxolone. In an exemplary embodiment, the methods of the invention are performed in vivo.

In one embodiment, methods for treating immune conditions characterized by ATP-mediated T cell activation are provided. In one embodiment, these methods comprise administering to a mammalian subject at least one of the inhibitors of ATP-mediated T cell activation disclosed herein, either alone or in conjunction with alternative immunotherapeutic or immunosuppressive protocols. In a preferred embodiment, at least one inhibitor of ATP-mediated T cell activation is administered to a subject, wherein said inhibitor is capable of interfering with the interaction of ATP and a P2 receptor, e.g., a P2X receptor, e.g., a T cell P2X7 receptor, and inhibiting ATP signaling. In an exemplary embodiment, the inhibitor of ATP-mediated T cell activation is oATP.

Induction of Treq Cell Differentiation and/or Expansion

The invention provides methods for treating immune conditions that benefit from the differentiation and/or expansion of Treg cells. In one embodiment, these methods comprise administering to a mammalian subject Treg cells that have been differentiated and/or expanded by contact with at least one agent disclosed herein, either alone or in conjunction with alternative immunotherapeutic or immunosuppressive protocols. In a preferred embodiment, the Treg cells are differentiated and/or expanded in vitro according to methods of the present invention and subsequently administered to the subject.

In a preferred embodiment, the agent that induces Treg cell differentiation and/or expansion is a composition comprising oATP. The composition preferably also comprises (1) a T cell primary stimulator; and (2) a cellular component or soluble mediator.

In certain embodiments, the T cell primary stimulator is a ligand (e.g., CD3 or anti-CD3) that binds to the T cell receptor (TCR) and initiates a primary stimulation signal. T cell primary stimulators include other natural and synthetic ligands. A natural ligand can include MHC with or without a peptide presented. Other ligands can include, but are not limited to, a peptide, polypeptide, growth factor, cytokine, chemokine, glycopeptide, soluble receptor, steroid, hormone, mitogen such as PHA, other superantigens, peptide-MHC complexes and soluble MHC complexes. In other embodiments, the T cell stimulator works by an alternate mechanism. Such stimulators include, e.g., protein kinase C activators, such as phorbol esters (e.g., phorbol myristate acetate), and calcium ionophores (e.g., ionomycin, which raises cytoplasmic calcium concentrations). The use of such agents bypasses the TCR/CD3 complex but delivers a stimulatory signal to T cells.

In certain embodiments, the cellular component or soluble mediator comprises one or more cell products selected from, e.g., antigen-presenting cell (e.g., from autologous, syngeneic, allogeneic, or xenogeneic irradiated splenocytes), mobilized cell products, including but not limited to leukopheresis cell products, and/or bone marrow derived cell products such as, for example, iliac crest cell products and/or vertebral bodies, as well as cell products from other sources of lymphoid tissues such as from lymph nodes and spleen. In other embodiments, the cellular component or soluble mediator comprises one or more soluble agents selected from retinoic acid (see, e.g., Hill et al., Immunity 29:758-770 (2008)), rapamycin, inhibitors of DNA methylation (e.g., 5-azacytidine; see, e.g,. Kim and Leonard, J. Exp. Med. 204(7):1543-1551 (2007)), cytokines (e.g., TGF-beta and interleukin (IL)-2), inhibitors of histone deacetylase (HDAC) such as trichostatin A (Taoet al., Nature Medicine 13:1299 (2007)) and α1-antitrypsin (Lewis et al, PNAS 105: 16236 (2008)).

The composition may also comprise other agents, including but not limited to: CD80, 4-1 BB, CD52 agonists, CD28 antibodies, lymphocyte function associated antigen-3 (LFA-3), CD2, CD40, CD80/B7-1, CD86/B7-2, OX-2, CD70, and CD82.

In an exemplary embodiment, the composition comprises oATP, anti-CD3 antibody, and syngeneic irradiated splenocytes. The composition may further comprise IL-2.

In one embodiment, the composition induces Treg cell differentiation/expansion more effectively than a composition comprising oATP, anti-CD3 antibody, and anti-CD28 antibody.

T cells to be differentiated and/or expanded can be obtained from, e.g., mammalian sources such as a human, dog, cat, mouse, rat, or transgenic species thereof. T cells, e.g., naïve T cells or Treg cells, can be isolated from, e.g., peripheral blood mononuclear cells, bone marrow, lymph node tissue, cord blood, thymus tissue, tissue from a site of infection, spleen tissue, tumors or T cell lines.

Cells capable of differentiating into Treg cells include mammalian progenitor cells such as naïve T cells, e.g., naïve CD4⁺ T cells. The invention also contemplates the use of the agents and methods of the invention to reverse other types of T cells, e.g., Th17 cells, to the Treg lineage. Methods of using the agents of the invention to reverse a pathogenic T cell phenotype to a protective T cell phenotype are thus provided. The invention also provides methods of using the agents of the invention to maintain a Treg cell phenotype, for example, by inhibiting conversion of Treg cells into non-Treg cell types (e.g., pathogenic T cell types such as Th17 cells).

Methods of obtaining T cells from the aforementioned sources are known in the art. For example, T cells can be obtained from extracted blood using FICOLL™ separation. Alternatively, T cells can be obtainaed from the circulating blood of a subject by apheresis or leukapheresis. Enrichment and/or isolation of specific subpopulations of T cells, e.g., naïve CD4⁺ cells and/or Treg cells, may be performed using positive and negative selection techniques known in the art, including but not limited to: fluorescence activated cell sorting (FACS), magnetic separation using antibody-coated magnetic beads, affinity chromatography, cytotoxic agents joined to a monoconal antibody or used in conjunction with a monoclonal antibody, e.g. complement and cytotoxins, and “panning” with antibody attached to a solid matrix, e.g., plate, or other convenient technique. Positive selection may be combined with negative selection against T cells comprising surface makers specific to non-desired T cell types.

In some embodiments, naïve CD4⁺ cells are purified for subsequent differentiation into Treg cells. Naïve CD4+cells can be isolated using, e.g., negative selection to remove CD8⁺ T cells and effector memory T cells (CD54R0), B cells (CD19), macrophages, natural killer cells, and neutrophils. In preferred embodiments, at least 55%, 65%, 75%, 85%, 90%, 95%, 98% or 100% of the cells of the composition resulting from the aforementioned selection techniques are naïve CD4⁺ cells.

Treg cells comprise cells that are CD4⁺CD25⁺ and are also characterized by expression of cytotoxic T lymphocyte antigen (CTLA)-4, glucocorticoid-induced TNF receptor (GITR) and the forkhead/winged-helix transcription factor Foxp3. Treg cells can be isolated from a mixed population of cells (e.g., from a population comprising peripheral blood mononuclear cells, or from a population comprising a mixture of differentiated Treg cells and naïve CD4⁺ cells) based on expression of CD25, GITR, CTLA-4, and Foxp3 using methods known in the art, In some embodiments, regulatory T cells will be separated from other cells by removing all cells that express CD127, which is down regulated in regulatory T cells. In one embodiment, the regulatory T cells may be selected against dead cells by employing dyes that specifically associate with dead cells (e.g., propidium iodide, ethidium monoazaide). In other embodiments, the regulatory T cells are obtained using other methods known in the art, e.g., as described in U.S. Patent Publication Nos. 20060063256, 20060233751, 20060240024 and 20080279834, which are incorporated herein by reference in their entirety. In preferred embodiments, at least 55%, 65%, 75%, 85%, 90%, 95%, 98% or 100% of the cells of the composition resulting from the aforementioned selection and isolation techniques are regulatory T cells.

In one embodiment, CD4⁺CD25⁺Foxp3⁺ Treg cells are isolated directly from peripheral blood samples in a two-step method. The first step involves enrichment of CD4⁺ T cells by negative selection of undesired cells using RosetteSep® technology, which cross-links red blood cells to unwanted cells in the sample. The cross-linked cells can then be pelleted by centrifugation over density medium and discarded. The second step involves positive selection of CD25^{+bright} cells from the enriched CD4⁺ T cell population by column-free immunomagnetic selection. This step may be automated using, e.g., RoboSept®, a pipetting robot with true walk-away capability. This two-step process significantly shortens the time required to obtain highly purified Treg samples from whole blood. The process is described in detail in, e.g., U.S. Pat. No. 7,135,335, incorporated herein by reference in its entirety. Any of a number of other methods for separating highly purified populations of Treg cells known in the art may be used in accordance with the present invention.

In one embodiment, Treg cells are expanded using CD3/CD28 expansion beads with high doses of IL-2 and rapamycin. This expansion protocol is described in detail in, e.g., U.S. Patent Publication No. 20050196386, incorporated herein by reference in its entirety. Any of a number of other methods for expanding Treg cells known in the art may be used in accordance with the present invention.

Treg cell stimulation and expansion can be carried out in any cell culture environment, e.g., culture flasks, culture bags, or any container capable of holding cells (e.g., a bioreactor), preferably in a sterile environment. One or more components of the agent that induces Treg cell differentiation and/or expansion may be in soluble form or immobilized on a solid support, such as a bead (e.g., a paramagnetic bead) or tissue culture dish. The solid phase surface can be plastic, glass, or any other suitable material. The stimulation and expansion can take place in one or more stages of cell culturing (see, e.g., U.S. Patent Publication No. 20060286067). The Treg cells are preferably expanded at least 2-fold, and more preferably at least 10, 50, 100, 200, 300, 500, 800, or 1000-fold.

Once isolated, differentiated, and/or expanded, Treg cells can be characterized based on expression of Foxp3, as well as by production of TGF-beta and failure to produce IL-2, IL-10, IL-4, IL-5, and IFN-gamma. The suppressive activity of the isolated Treg cells can be tested by, e.g., coculture with responder T cells. The present invention also envisages manipulating the expanded cells, for example through cytokine stimulation or by adding genes or interest, such as therapeutic genes or knock-out genes, prior to administration to a subject. For example, Treg cells may serve as a “Trojan Horse” to deliver suppressive or other biologic factors to sites of inflammation, e.g., IL-4, stem cell growth factors, angiogenesis regulators, genetic deficiencies, etc. Further, overexpression of Foxp3 has been shown to transform otherwise pathogenic T cells into regulatory T cells, and polyclonally expanded T cells can be transduced with genes encoding an antigen-specific TCR plus Foxp3 to generate potent antigen-specific regulatory T cells in very high numbers. These antigen-specific approaches decrease the requirement for high initial cell numbers while maximizing regulatory T cell specificity and function.

### Methods of Modulating T Cell-Dependent Immune Responses

The present invention is generally directed to methods and compositions for modulating T cell activity, having multiple therapeutic applications for immunological tolerance, autoimmunity, immunosuppression, and immunotherapy. In particular, the present invention provides methods of (1) inhibiting at least one T cell activity, comprising the step of contacting a T cell with an agent that inhibits ATP-mediated T cell activation, and (2) modulating at least one Treg cell activity, e.g., Treg cell differentiation, expansion, and/or immune suppressive activity, comprising the step of contacting a Treg cell with an agent that modulates at least one Treg cell activity. In certain embodiments, method (1) is performed in vivo; and method (2) is performed in vitro and is followed by in vivo administration of the Treg cells with modulated activity (e.g., differentiated and/or expanded Treg cells, or Treg cells with enhanced immune suppressive activity).

As disclosed for the first time herein, ATP released from T cells in the course of activation results in autocrine activation of P2X receptors with MAPK activation. This represents an essential costimulatory factor for productive T cell activation and expansion.

Also as disclosed for the first time herein, pannexin is expressed on T cells and acts as a positive regulator of T cell activity, wherein signaling mediated by ATP results in pannexin hemichannel formation, leading to T cell activation.

Thus, ATP signaling is responsible for promoting T cell responses, such as cell cycle progression, differentiation, survival, cytokine production and cytolytic activation. These findings enable the use of therapeutic agents capable of interfering with ATP-mediated T cell activation (e.g., P2X receptor antagonists or agents that block pannexin hemichannel formation, or agents that block channels associated with ATP-mediated T cell activation) to modulate T cell activity (e.g., helper T cell activity) for the purpose of treating, among other conditions, autoimmune conditions and transplant-related immune responses.

As disclosed for the first time herein, the differentiation of Th17 cells (T cells which secrete IL-17) is inhibited by oATP. This T cell subset is currently viewed as a major component of T lymphocyte-dependent inflammatory responses. Accordingly, the present invention provides methods for inhibiting differentiation of Th17 cells and/or secretion of IL-17 by contacting a Th17 cell with at least one inhibitor of ATP-mediated T cell activation. The present invention also provides methods for inhibiting the conversion of protective T cells, e.g,. Treg cells, to Th17 cells, and for promoting the conversion of Th17 cells to protective T cells, e.g., to a Treg lineage. Said methods of the invention may be performed, e.g., in vitro or in vivo.

Further, as disclosed for the first time herein, oATP in conjunction with anti-CD3, IL-2 and syngeneic irradiated splenocytes induces both differentiation and significant expansion of active regulatory T cells (Treg cells) with high expression levels of Foxp3. Thus, oATP, particularly oATP in compositions comprising certain other agents, can induce Treg differentiation and/or expansion. These findings enable the use of compositions comprising oATP to modulate T cell activity (e.g., regulatory T cell activity) for the purpose of treating, among other conditions, autoimmune conditions and transplant-related immune responses (e.g., graft-versus-host disease).

The present invention provides novel uses for inhibitors of ATP-mediated T cell activation, and inducers of Treg differentiation and/or expansion, for use in treating inflammatory and autoimmune conditions. The inhibitors and inducers of the present invention can be used to modulate, agonize, block, increase, inhibit, reduce, antagonize or neutralize the activity of T cells in the treatment of specific conditions such as asthma, allergies, rheumatoid arthritis, psoriatic arthritis, arthritis, endotoxemia, type I diabetes, inflammatory bowel disease (IBD), colitis, multiple sclerosis, transplant rejection, graft-versus-host disease, amyotrophic lateral sclerosis, demyelinating disorders, scleroderma, Sjogren syndrome, Erdheim-Chester syndrome, Crohn's Disease syndrome, Takayasu arteritis, sarcoidosis, autoimmune hemolytic anemia, Werlhof's idiopathic thrombopenic syndrome, psoriasis, cutaneous T-cell lymphoma, cutaneous graft-versus-host disease, atopic dermatitis, allergic contact dermatitis, alopecia areata, vitiligo, drug-related eruptions, contact hypersensitivity, lupus erythematosus, pityriasis lichenoides et varioliformis, pityriasis lichenoides chronica, eczema, lichen planus, and any of a number of other immune conditions disclosed herein or known in the art. Such inhibitors and inducers will be beneficial for any T cell-mediated immune condition known now or later discovered. In a preferred embodiment, said inhibitors and inducers comprise oATP.

In one embodiment, the methods of the invention are used to treat an inflammatory condition wherein said inflammatory condition is not an innate immune system inflammatory condition, or wherein said inflammatory condition is not entirely an innate immune system inflammatory condition. In certain embodiments, the methods of the invention are used to treat an inflammatory condition wherein said inflammatory condition is partially or completely an adaptive immune system inflammatory condition. The innate immune system comprises cells and mechanisms that respond to pathogens in a non-specific manner. Inflammation caused by cells of the innate immune system generates mediators (e.g., histamine, bradykinin, serotonin, leukotrienes, and prostaglandins) that sensitize pain receptors, cause vasodilation of the blood vessels, and attract phagocytes. These reactions are not directly involved in the pathogenic response of autoimmune disease. In contrast, the adaptive immune system, which comprises T lymphocyte-dependent inflammatory responses, responds to pathogens in an antigen-specific manner, and can be directly involved in mediation of autoimmune disease. The cells and processes of the innate immune system are distinct from the cells and processes of the adaptive immune system. In certain embodiments, the methods of the invention are used to treat an adaptive immune system inflammatory and/or autoimmune condition. In one embodiment, the methods of the invention are used to treat the T cell activation-related initiating phase of an inflammatory and/or autoimmune process. In an exemplary embodiment, said methods of the invention are performed in vivo.

In a preferred embodiment, methods for suppressing a host immune response to antigenic stimulation are provided, comprising the administration to the host of at least one of the aforementioned inhibitors of ATP-mediated T cell activation, one of the aforementioned agents that induce Treg cell differentiation and/or expansion, and/or Treg cells differentiated and/or expanded by contact with one of the aforementioned agents. For example, the antigenic stimulation may be from self antigens in the context of autoimmune disease, or from donor antigens present in transplanted organs and tissues. In an exemplary embodiment, said methods are performed in vivo.

The present invention also provides methods for treating an inflammatory or immune condition using pharmaceutical compositions comprising a pharmaceutically acceptable carrier and at least one inhibitor of ATP-mediated T cell activation and/or one inducer of Treg cell differentiation and/or expansion. In some embodiments, the pharmaceutical composition comprises the Treg cells differentiated and/or expanded by said inducer. In an exemplary embodiment, said methods of treatment are performed in vivo.

Methods of the invention may be useful in the treatment of, for example, inflammatory bowel disease (e.g., Crohn's disease and celiac disease), irritable bowel syndrome, migraine, headache, low back pain, fibromyalgia, myofascial disorders, viral infections (e.g. hepatitis C and, particularly, influenza, common cold, herpes zoster, and AIDS), autoimmune hepatitis, bacterial infections, fungal infections, dysmenorrhea, burns, surgical or dental procedures, malignancies (e.g. breast cancer, colon cancer, and prostate cancer), atherosclerosis, enterogenic spondyloarthropathies, gout, arthritis, osteoarthritis, juvenile arthritis, rheumatoid arthritis, psoriatic arthritis, fever (e.g. rheumatic fever), ankylosing sodalities, systemic lupus erythematosus (SLE), vasculitis, pancreatitis, nephritis, bursitis, conjunctivitis, iritis, scleritis, uveitis, wound healing, dermatological conditions (e.g., psoriasis, cutaneous T-cell lymphoma, cutaneous graft-versus-host disease, atopic dermatitis, allergic contact dermatitis, alopecia areata, vitiligo, drug-related eruptions, contact hypersensitivity, lupus erythematosus, pityriasis lichenoides et varioliformis, pityriasis lichenoides chronica, eczema, and lichen planus), stroke, diabetes mellitus, neurodegenerative disorders such as Alzheimer's disease and multiple sclerosis, autoimmune diseases (e.g,. amyotrophic lateral sclerosis, demyelinating disorders, scleroderma, Sjogren syndrome, Erdheim-Chester syndrome, Crohn's Disease syndrome, Takayasu arteritis, autoimmune hemolytic anemia and Werlhof's idiopathic thrombopenic syndrome), osteoporosis, asthma, chronic obstructive pulmonary disease, pulmonary fibrosis, allergic disorders, rhinitis, ulcers, coronary heart disease, sarcoidosis, transplant rejection, graft versus host disease and any other disease with an immune or inflammatory component. In certain embodiments, the methods of the invention are used to treat a disease with an adaptive immune component.

In a further aspect, methods for treating T lymphocyte-dependent inflammatory or immune conditions are provided. In a preferred embodiment, the T lymphocyte-dependent inflammatory or immune condition is selected from the group consisting of, for example, asthma, allergies, rheumatoid arthritis, psoriatic arthritis, arthritis, endotoxemia, type I diabetes, inflammatory bowel disease (IBD), colitis, multiple sclerosis, transplant rejection, graft-versus-host disease, amyotrophic lateral sclerosis, demyelinating disorders, scleroderma, Sjogren syndrome, Erdheim-Chester syndrome, Crohn's Disease syndrome, Takayasu arteritis, sarcoidosis, autoimmune hemolytic anemia, Werlhof's idiopathic thrombopenic syndrome, and dermatological conditions (e.g., psoriasis, cutaneous T-cell lymphoma, cutaneous graft-versus-host disease, atopic dermatitis, allergic contact dermatitis, alopecia areata, vitiligo, drug-related eruptions, contact hypersensitivity, lupus erythematosus, pityriasis lichenoides et varioliformis, pityriasis lichenoides chronica, eczema, and lichen planus). In certain embodiments, the inflammatory or immune condition is associated with degranulation of mastocytes. In a preferred emodiment, said methods are performed in vivo. In one embodiment, the methods comprise administering to a subject, e.g., a mammalian subject, at least one of the aforementioned inhibitors of ATP-mediated T cell activation, one of the aforementioned agents that induce Treg cell differentiation and/or expansion, and/or Treg cells differentiated and/or expanded by contact with one of the aforementioned agents, either alone or in conjunction with alternative immunotherapy and/or immunosuppressive agents and/or protocols.

In one embodiment, methods for improving the outcome of organ and tissue transplantation and prolonging graft survival are provided. In one embodiment, these methods comprise administering to a transplant recipient at least one of the aforementioned inhibitors of ATP-mediated T cell activation, one of the aforementioned agents that induce Treg cell differentiation and/or expansion, and/or Treg cells differentiated and/or expanded by contact with one of the aforementioned agents, either alone or in conjunction with alternative immunotherapy and/or immunosuppressive protocols. In a preferred embodiment, at least one inhibitor of ATP-mediated T cell activation is administered to the transplant recipient, wherein administration of said inhibitor is effective to decrease the recipient immune response, e.g., T cell activation, against donor antigens present in the graft. In another preferred embodiment, at least one agent that induces Treg cell differentiation and/or expansion is administered to the transplant recipient, wherein administration of said agent is effective to suppress the recipient immune response against donor antigens present in the graft. In still another preferred embodiment, Treg cells differentiated and/or expanded by contact with said inducing agent are administered to the transplant recipient, wherein administration of said Treg cells is effective to suppress the recipient immune response against donor antigens present in the graft. See, e.g., Taylor et al., Blood 99:3493-3499 (2002). In one embodiment, the graft is an allograft. In another embodiment, the graft is a xenograft.

In a particularly preferred embodiment, the methods of the invention may be used to prolong the survival of grafted tissue. Preferred compositions for use in the prevention of acute and/or chronic graft rejection comprise ATP antagonists, e.g., oATP, and/or agents that block pannexin hemichannel assembly, e.g., PX10. Especially preferred agents include small molecule chemical compositions that mimic the natural interaction of ATP and receptors for ATP. Compositions that induce Treg cell differentiation and/or expansion preferably also comprise (1) a T cell primary stimulator; and (2) a cellular component or a soluble mediator. The methods of the invention may be performed in vitro or in vivo.

In one embodiment, at least one agent that modulates a T cell-dependent immune response is administered to the recipient of an implant consisting of biological material contained in a device, to decrease the recipient immune response and/or to prolong the survival of grafted tissue. The device may be any device for the implantation of biological material in a patient, e.g., the device as described in U.S. Patent Publication No. 2006/0024276, U.S. Patent No. 6,716,246 or PCT Patent Publication No. WO 08/097498, each of which is incorporated herein by reference in its entirety. In one embodiment, at least one agent that modulates a T cell-dependent immune response is administered to the recipient of said device locally, i.e. at or in the vicinity of the site of device implantation. Administration of an agent “in the vicinity of” a site, as used herein, refers to administration of the agent to cells or tissues that are in cellular communication with said site. In one embodiment, at least one agent that modulates a T cell-dependent immune response is administered to the recipient of said device at one or more lymph nodes near or surrounding the site of device implantation.

In one embodiment, at least one agent that modulates a T cell-dependent immune response is administered to the recipient of an implant comprising a device for the administration of a substance, e.g., to decrease a recipient immune or inflammatory response. The device may be any device for the implantation of biological material in a patient, e.g., the device as described in U.S. Patent Publication No. 2006/0024276, U.S. Pat. No. 5,324,518, U.S. Pat. No. 6,716,246 or PCT Patent Publication No. WO 08/097498, each of which is incorporated herein by reference in its entirety. In one embodiment, at least one agent that modulates a T cell-dependent immune response is administered to the recipient of said device locally, i.e. through the device, or at or in the vicinity of the site of device implantation. In one embodiment, at least one agent that modulates a T cell-dependent immune response is administered to the recipient of said device at one or more lymph nodes near or surrounding the site of device implantation.

Also provided are methods for inhibiting T cell activity, e.g., T cell activation, proliferation, and effector function, using a pharmaceutical composition that comprises inhibitors of ATP-mediated T cell activation and at least one other immunosuppressive or anti-inflammatory agent. In an exemplary embodiment, said methods of the invention are performed in vivo.

Also provided are methods for increasing Treg cell immune suppressive activity, using a pharmaceutical composition that comprises oATP and at least one other immunosuppressive or anti-inflammatory agent. In some embodiments, the pharmaceutical composition also comprises (1) a T cell primary stimulator; and (2) a cellular component or a soluble mediator. In an exemplary embodiment, T cells (e.g., naïve T cells) are contacted by the aforementioned composition in vitro and differentiated, and the resulting Treg cells expanded into a population of Treg cells that are then administered in a pharmaceutical composition that may comprise at least one other immunosuppressive or anti-inflammatory agent, or at least one agent that helps to promote and maintain the Treg phenotype in an antigen-specific fashion, for in vivo administration to a subject in need thereof. In another embodiment, oATP (alone or in conjunction with other agents) is administered to a subject in need thereof to increase Treg cell immune suppressive activity in vivo.

Immunosuppressive agents may comprise one or more of, e.g., cyclosporine, rapamycin, campath-1H, ATG, Prograf, anti IL-2r, MMF, FTY, LEA, interferon, interleukin-2, cyclosporin A, diftitox, denileukin, levamisole, azathioprine, brequinar, gusperimus, 6-mercaptopurine, mizoribine, rapamycin, tacrolimus (FK-506), folic acid analogs (e.g., denopterin, edatrexate, methotrexate, piritrexim, pteropterin, Tomudex®, and trimetrexate), purine analogs (e.g., cladribine, fludarabine, 6-mercaptopurine, thiamiprine, and thiaguanine), pyrimidine analogs (e.g., ancitabine, azacitidine, 6-azauridine, carmofur, cytarabine, doxifluridine, emitefur, enocitabine, floxuridine, fluorouracil, gemcitabine, and tegafur) fluocinolone, triaminolone, anecortave acetate, fluorometholone, medrysone, prednislone, etc.

Anti-inflammatory agents may comprise one or more of, e.g., NSAIDs, interleukin-1 antagonists, dihydroorotate synthase inhibitors, p38 MAP kinase inhibitors, TNF-α inhibitors, TNF-α sequestration agents, and methotrexate. More specifically, anti-inflammatory agents may comprise one or more of, e.g., anti-TNF-α, lysophylline, alpha 1-antitrypsin (AAT), interleukin-10 (IL-10), pentoxyfilline, COX-2 inhibitors, 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, aminoarylcarboxylic acid derivatives (e.g., enfenamic acid, etofenamate, flufenamic acid, isonixin, meclofenamic acid, mefenamic acid, niflumic acid, talniflumate, terofenamate, tolfenamic acid), arylacetic acid derivatives (e.g., aceclofenac, acemetacin, alclofenac, amfenac, amtolmetin guacil, bromfenac, bufexamac, cinmetacin, clopirac, diclofenac sodium, etodolac, felbinac, fenclozic acid, fentiazac, glucametacin, ibufenac, indomethacin, isofezolac, isoxepac, lonazolac, metiazinic acid, mofezolac, oxametacine, pirazolac, proglumetacin, sulindac, tiaramide, tolmetin, tropesin, zomepirac), arylbutyric acid derivatives (e.g., bumadizon, butibufen, fenbufen, xenbucin), arylcarboxylic acids (e.g., clidanac, ketorolac, tinoridine), arylpropionic acid derivatives (eg., alminoprofen, benoxaprofen, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprolen, pirprofen, pranoprofen, protizinic acid, suprofen, tiaprofenic acid, ximoprofen, zaltoprofen), pyrazoles (e.g., difenamizole, epirizole), pyrazolones (e.g., apazone, benzpiperylon, feprazone, mofebutazone, morazone, oxyphenbutazone, phenylbutazone, pipebuzone, propyphenazone, ramifenazone, suxibuzone, thiazolinobutazone), salicylic acid derivatives (e.g., acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide o-acetic acid, salicylsulfuric acid, salsalate, sulfasalazine), thiazinecarboxamides (e.g., ampiroxicam, droxicam, isoxicam, lornoxicam, piroxicam, tenoxicam), epsilon.-acetamidocaproic acid, s-adenosylmethionine, 3-amino-4-hydroxybutyric .acid, amixetrine, bendazac, benzydamine, α-bisabolol, bucolome, difenpiramide, ditazol, emorfazone, fepradinol, guaiazulene, nabumetone, nimesulide, oxaceprol, paranyline, perisoxal, proquazone, superoxide dismutase, tenidap, zileuton, candelilla wax, alpha bisabolol, aloe vera, Manjistha, Guggal, kola extract, chamomile, sea whip extract, glycyrrhetic acid, glycyrrhizic acid, oil soluble licorice extract, monoammonium glycyrrhizinate, monopotassium glycyrrhizinate, dipotassium glycyrrhizinate, 1-beta-glycyrrhetic acid, stearyl glycyrrhetinate, and 3-stearyloxy-glycyrrhetinic acid, disodium 3-succinyloxy-beta-glycyrrhetinate, etc.

Agents that help to promote and maintain the Treg phenotype may comprise one or more of, e.g., oATP, TGF-beta, retinoic acid, rapamycin, 5-azacytidine, and trichostatin A.

It is further contemplated that the subject compositions and methods may be synergistically combined with immunotherapies based on modulation of other T cell costimulatory pathways, such as with CD28, ICOS, PD-1, CTLA-4 and/or BTLA modulation, for example.

### Pharmaceutical Formulations

Methods of the invention may comprise administration of a therapeutic agent alone, but preferably comprise administration by way of known pharmaceutical formulations, including tablets, capsules or elixirs for oral administration, suppositories for rectal administration, sterile solutions or suspensions for parenteral or intramuscular administration, liposomal or encapsulated formulations, formulations wherein the therapeutic agent is alone or conjugated to a delivery agent or vehicle, and the like.

Such formulations may be prepared in accordance with standard and/or accepted pharmaceutical practice.

It will be appreciated that therapeutic entities of the invention will be administered with suitable carriers, excipients, and/or other agents that are incorporated into formulations to provide improved transfer, delivery, tolerance, and the like. A multitude of appropriate formulations can be found in the formulary known to all pharmaceutical chemists: Remington's Pharmaceutical Sciences (15^{th} ed, Mack Publishing Company, Easton, Pa. (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present invention, provided that the active ingredient in the formulation is not inactivated by the formulation and the formulation is physiologically compatible and tolerable with the route of administration. See also Powell et al. “Compendium of excipients for parenteral formulations” PDA J Pharm Sci Technol 52:238-311 (1998) and the citations therein for additional information related to excipients and carriers well known to pharmaceutical chemists.

In one embodiment, the therapeutic agent is administered in a topical formulation. Topical forms of administration may consist of, for example, aqueous and nonaqueous gels, creams, multiple emulsions, microemulsions, liposomes, ointments, aqueous and nonaqueous solutions, lotions, aerosols, skin patches, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer.

Topical formulations of the invention may include a dermatologically acceptable carrier, e.g., a substance that is capable of delivering the other components of the formulation to the skin with acceptable application or absorption of those components by the skin. The carrier will typically include a solvent to dissolve or disperse the therapeutic agent, and, optionally one or more excipients or other vehicle ingredients. Carriers useful in accordance with the topical formulations of the present invention may include, by way of non-limiting example, water, acetone, ethanol, ethylene glycol, propylene glycol, butane-1,3-diol, acrylates copolymers, isopropyl myristate, isopropyl palmitate, mineral oil, butter(s), aloe, talc, botanical oils, botanical juices, botanical extracts, botanical powders, other botanical derivatives, lanolin, urea, petroleum preparations, tar preparations, plant or animal fats, plant or animal oils, soaps, triglycerides, and keratin(s). Topical formulations of the invention are prepared by mixing a compound of the invention with a topical carrier according to well-known methods in the art, for example, methods provided by standard reference texts e.g., Remington: The Science and Practice of Pharmacy, 1577-1591, 1672-1673, 866-885 (Alfonso R. Gennaro ed. 19th ed. 1995); and Ghosh et al., Transdermal and Topical Drug Delivery Systems (1997).

Additionally, moisturizers or humectants, sunscreens, fragrances, dyes, and/or thickening agents such as paraffin, jojoba, PABA, and waxes, surfactants, occlusives, hygroscopic agents, emulsifiers, emollients, lipid-free cleansers, antioxidants and lipophilic agents, may be added to the topical formulations of the invention if desired.

A topical formulation of the invention may be designed to be left on the skin and not washed shortly after application. Alternatively, the topical formulation may be designed to be rinsed off within a given amount of time after application.

Methods of Delivery

Methods of the invention may comprise administration of a therapeutic agent, alone or in the form of a composition, by any desirable means, e.g., orally, intravenously, intramuscularly, intraperitoneally, intrathecally, alimentarily, intraspinally, intra-articularly, intra-joint, subcutaneously, buccally, vaginally, rectally, dermally, transdermally, ophthalmically, auricularly, mucosally, nasally, tracheally, bronchially, sublingually, intranodally, by any parenteral route or via inhalation, in a pharmaceutically acceptable dosage form.

In one embodiment, the therapeutic agent is administered directly to its site of therapeutic activity, e.g., the lymph nodes. For example, the therapeutic agent may be injected directly into the lymph nodes. Preferred lymph nodes for intranodal injections of inhibitors of T cell-dependent activation are the major lymph nodes located in the regions of the groin, the underarm and the neck. In another embodiment, the therapeutic agent is administered distal to the site of its therapeutic activity.

In one embodiment, the therapeutic agent is administered topically. In certain of these embodiments, the therapeutic agent is administered topically to treat a dermatological condition. While the amount of the topical formulation to be applied will depend upon, for example, the intended usage of the final composition, i.e., therapeutic versus maintenance regimen, and sensitivity of the individual subject to the formulation, in one embodiment the topical formulations of the invention are applied to affected body parts at regular intervals. In certain embodiments, the composition is applied more frequently during the initial stages of treatment until the desired effect is achieved, then less frequently when maintenance is desired.

The topical formulations of the present invention may be applied by various methods. In one embodiment, the formulation is applied to the area of the skin affected with inflammation. The area is then massaged or rubbed until the formulation is distributed evenly or disappears. The process can be repeated 1, 2, 3, 4, 5, 6 or more times in a day. In another method, the topical formulation is applied to a dermal patch, which is then mounted onto the affected area of the skin for 30 minutes to several hours, days, weeks, or more. In another embodiment, the topical formulation of the present invention is delivered to the affected area using iontophoresis. In this method, the topical formulation is placed in a container or a patch that is connected to an electrode. The container is then placed on the affected area, and the electrode is activated. This leads to the generation of a current that delivers the topical formulation through the skin by electrical repulsion.

Other suitable methods for delivering the topical formulations of the present invention through the skin include phonophoresis and cellophane wrapping. In phonophoresis, the topical formulation is first applied to the affected area on the skin. An ultrasound apparatus is then placed on the affected area. Once activated, the apparatus delivers the formulation through the skin by ultrasonic energy. In cellophane wrapping, the formulation is applied to the affected area and wrapped with a cellophane film anywhere from several minutes to several hours, days, weeks, or months.

In one embodiment of the invention, one or more agents of the invention are nanoencapsulated into nanoparticles for delivery. The nanoencapsulation material may be biodegradable or nondegradable. The nanoencapsulation materials may be made of synthetic polymers, natural polymers, oligomers, or monomers. Synthetic polymers, oligomers, and monomers include those derived from polyalkyleneoxide precursor molecules, such as poly(ethylene oxide) (PEO), poly(ethylene glycol) (PEG) and copolymers with poly(propylene oxide) (PEG-co-PPO), poly (vinyl alcohol) (PVA), poly(vinylpyrrolidone) (PVP), poly(ethyloxazoline) (PEOX), polyaminoacids, and pseudopolyamino acids, and copolymers of these polymers. Sawhney et al., Macromolecules 26:581-587 (1993). Copolymers may also be formed with other water-soluble polymers or water insoluble polymers, provided that the conjugate is water soluble. An example of a water-soluble conjugate is a block copolymer of polyethylene glycol and polypropylene oxide, commercially available as a Pluronic.TM. surfactant (BASF).

Natural polymers, oligomers and monomers include proteins, such as fibrinogen, fibrin, gelatin, collagen, elastin, zein, and albumin, whether produced from natural or recombinant sources, and polysaccharides, such as agarose, alginate, hyaluronic acid, chondroitin sulfate, dextran, dextran sulfate, heparin, heparin sulfate, heparan sulfate, chitosan, gellan gum, xanthan gum, guar gum, water soluble cellulose derivatives, and carrageen. These polymers are merely exemplary of the types of nanoencapsulation materials that can be utilized and are not intended to represent all the nanoencapsulation materials within which entrapment is possible.

“Controlled release” refers to the release of an agent such as a drug from a composition or dosage form in which the agent is released according to a desired profile over an extended period of time. Controlled release profiles include, for example, sustained release, prolonged release, pulsatile release, and delayed release profiles. In contrast to immediate release compositions, controlled release compositions allow delivery of an agent to a subject over an extended period of time according to a predetermined profile. Such release rates can provide therapeutically effective levels of agent for an extended period of time and thereby provide a longer period of pharmacologic or diagnostic response as compared to conventional rapid release dosage forms. Such longer periods of response provide for many inherent benefits that are not achieved with the corresponding short acting, immediate release preparations. For example, in the treatment of chronic pain, controlled release formulations are often highly preferred over conventional short-acting formulations.

Controlled release pharmaceutical compositions and dosage forms are designed to improve the delivery profile of agents, such as drugs, medicaments, active agents, diagnostic agents, or any substance to be internally administered to an animal, including humans. A controlled release composition is typically used to improve the effects of administered substances by optimizing the kinetics of delivery, thereby increasing bioavailability, convenience, and patient compliance, as well as minimizing side effects associated with inappropriate immediate release rates such as a high initial release rate and, if undesired, uneven blood or tissue levels.

Prior art teachings of the preparation and use of compositions providing for controlled release of an active compound provide various techniques for extending the release of a drug following administration. Exemplary controlled release formulations known in the art include specially coated pellets, microparticles, nanoparticles, implants, tablets, minitabs, and capsules in which the controlled release of a drug is brought about, for example, through selective breakdown of the coating of the preparation, through release through the coating, through compounding with a special matrix to affect the release of a drug, or through a combination of these techniques. Some controlled release formulations provide for pulsatile release of a single dose of an active compound at predetermined periods after administration.

In one embodiment, a controlled release formulation comprising an agent that modulates at least one T cell-dependent immune response is targeted to a particular cell type or tissue. This may be accomplished, for example, by binding a target drug/target substance to the controlled release formulation to secure it from outer intervention in vivo or in vitro (e.g., in cell culture or ex vivo) until it is exposed at a desired target site, such as within a target cell, as described in U.S. Patent Publication No. 2007/0190160. In a preferred embodiment, the controlled release formulation is targeted to the lymph nodes of the subject. In another preferred embodiment, the controlled release formulation is targeted to a site at or in the vicinity of a transplant (e.g., a transplanted organ) or an implant (e.g., an implanted device containing biological material).

Methods of the invention may comprise the step of administering the therapeutic agent at varying doses. Oral, pulmonary and topical dosages may range from between about 0.01 mg/kg of body weight per day (mg/kg/day) to about 100 mg/kg/day, preferably about 0.01 to about 10 mg/kg/day, and more preferably about 0.1 to about 5.0 mg/kg/day. For e.g. oral administration, the compositions typically contain between about 0.01 mg to about 500 mg, and preferably between about 1 mg to about 100 mg, of the active ingredient. Intravenously, the most preferred doses will range from about 0.001 to about 10 mg/kg/hour during constant rate infusion. Advantageously, therapeutic agents may be administered in a single daily dose, or the total daily dosage may be administered in divided doses of two, three or four times daily. In certain embodiments, the agent is administered for one, two, three, or four weeks, two months, three months, a year, two years, several years, or more, as determined to be suitable by the skilled practitioner.

The Treg cells differentiated and/or expanded by the methods of the present invention are useful for suppression of immune function in a subject. In particular, autologous cells can be isolated; modified, differentiated, and/or expanded in vitro; and subsequently administered or reimplanted in the subject. A therapeutically effective amount of Treg cells can be administered with a pharmaceutically acceptable carrier. Administration routes may include any suitable means, including, but not limited to, oral, rectal, vaginal, buccal, topical, nasal, dermal, transdermal, tracheal, sublingual, intranodal, parenteral, intravenous, intraperitoneal, injection, intranasal inhalation, lung inhalation, subcutaneous, ophthamlic, mucosal, auricular, intra-articular and intrathecal routes, and via the alimentary tract (for example, via the Peyers patches). Local routes of administration include intra-joint, intramuscular and intraspinal administration, as well as administration via vessels that drain into the pancreas such as the anterior and posterior pancreatico duodenal arteries, In preferred embodiments, Treg cells of the invention and pharmaceutical compositions comprising the cells are administered to the subject by intramuscular, intraperitoneal or intravenous injection, or by direct injection into the lymph nodes of the patient. In an exemplary embodiment, the Treg cells are administered locally to the site of inflammation. The route of administration selected will depend upon the particular treatment. The cells can be administered in a single dose or in several doses over selected time intervals in order to titrate the dose. In certain embodiments, from 10⁴/kg to 10⁹/kg treated cells, preferably from 10⁵/kg to 10⁷/kg cells, more preferably about 10⁶/kg cells are administered to the subject. The cells can be administered once or over a period of, e.g., 12, 24, 48, 72, or 96 hours; over a prolonged period of, e.g., one week, ten days, two weeks, one month, three months or six months; or for as long as the administration is of therapeutic benefit.

In the case of transplantation, the Treg cells may be alloactivated using the recipient cells. In a preferred embodiment, the Treg cells are differentiated and/or expanded in vitro in advance of the transplant surgery and administered during surgery to treat or prevent graft-versus-host disease. In certain embodiments, the Treg cells are administered using a controlled release mechanism, such as an artificial gel or clotted plasma.

In some embodiments, the Treg cells are administered to the subject in anticipation of an immune response-causing event such as a transfusion or a transplant. In this case, the regulatory T cells may be administered, e.g., one week or 6, 5, 4, 3, 2, or 1 day or less than 12, less than 4, or less than 2 hours prior to transfusion.

In any event, the physician, or the skilled person, will be able to determine the actual dosage that will be most suitable for an individual patient, which is likely to vary with the route of administration, the type and severity of the condition that is to be treated, as well as the species, age, weight, sex, renal function, hepatic function and response of the particular patient to be treated. The above-mentioned dosages are exemplary of the average case; there can, of course, be individual instances where higher or lower dosage ranges are merited, and such are within the scope of this invention.

In order that this invention may be better understood, the following examples are set forth. These examples are for purposes of illustration only and are not to be construed as limiting the scope of the invention in any manner.

### EXAMPLES

### Example 1

### Reduced Ca²⁺ in the ER and Increased Mitochondrial Ca²⁺ Uptake in crt^{-/-} T Cells

Previous studies have shown that CRT expression levels directly correlate with the amount of Ca²⁺ stored in the ER (Pinton et al., EMBO J 20:2690-2701 (2001); Arnaudeau et al., J Biol Chem 277:46696-46705 (2002)). We checked whether CRT exerted an analogous function in CD4⁺ T cell clones specific for the ovalbumin peptide 323-339 (OVAp). These clones were derived from FLC generated with hematopoietic progenitors from either crt^{+/+} or crt^{-/-} DO.11.10 TCR transgenic embryos (Porcellini et al., J Exp Med 203:461-471 (2006)). Cells were loaded with the Ca²⁺ indicator Fura-2 and stimulated with tapsigargin, an inhibitor of the sarcoplasmic endoplasmic reticulum Ca²⁺ ATPase (SERCA), in the absence of extracellular calcium. Quantification of the slow cytosolic Ca²⁺ elevation produced by passive Ca²⁺ leakage from the ER showed a 20% reduction in the ER Ca²⁺ content in crt^{-/-} cells compared to cre^{+/+} cells (FIG. 2A).

Ca²⁺ elevations following cell stimulation are sensed by mitochondria, which regulate Ca²⁺ signaling by buffering cytosolic Ca²⁺ (Rizzuto and Pozzan, Physiol Rev 86:369-408 (2006)). We investigated whether mitochondrial Ca²⁺ uptake in T cells was affected by crt deletion. We induced CCE in crt^{-/-} and crt^{+/+} T cells by depleting Ca²⁺ stores with tapsigargin in a Ca²⁺-free medium followed by addition of 0.5 mM Ca²⁺. After extensive washout of extracellular Ca²⁺, the Ca²⁺ ionophore ionomycin was added to release Ca²⁺ that accumulated in mitochondria upon CCE (Hoth et al., J Cell Biol 137:633-648 (1997)). FIG. 1A shows that Ca²⁺ accumulated to a greater extent in crt^{-/-} than crt^{+/+} cells, although the amplitude of cytosolic calcium elevation during CCE was similar in the two cell types. If ionomyin was added before the addition of 0.5 mM Ca²⁺, no differences were observed between crt^{-/-} and crt^{+/+} T cells (not shown), suggesting that Ca²⁺ was accumulated into mitochondria during CCE. This excludes constitutive mitochondrial Ca²⁺ overload in crt^{-/-} cells. Increasing extracellular calcium concentrations after Ca²⁺ store depletion was paralleled by an increase in mitochondrial calcium uptake in crt^{+/+}, but not crt^{-/-} cells (FIG. 1B). In fact, mitochondrial buffering remained constant in crt^{-/-} T cells within a range of 0.5-2 mM extracellular Ca²⁺.

Mitochondrial Ca²⁺ buffering influences Ca²⁺ dependent inactivation of CRAG channels by depleting Ca²⁺ ions in the proximity of the mouth of the channel (Gilabert et al., EMBO J 20:2672-2679 (2001); Hoth et al., Proc Natl Acad Sci USA 97:10607-10612 (2000); and Gilabert et al., EMBO J 19:6401-6407 (2000)). We exploited this property to further confirm that mitochondrial Ca²⁺ uptake was enhanced in crt^{-/-} T cells. We depleted tapsigargin-sensitive stores of Fura-2 loaded cells in a Ca²⁺-free medium and subjected the cells to two sequential additions of 0.5 mM Ca²⁺ (FIG. 1C and FIG. 2C). Since the rate of cytosolic Ca²⁺ rise depends on the activity of CRAG channels, quantification of this parameter at the first and the second addition of extracellular Ca²⁺ allows estimation of the degree of Ca2⁺-dependent inactivation of CRAG channels (Hoth et al., J Cell Biol 137:633-648 (1997)). In crt^{+/+} cells, we calculated a 57% reduction of CRAC activity at the second addition of extracellular Ca²⁺. However, in crt^{-/-} cells, CRAC channels showed only 14% inactivation at the second addition of extracellular calcium (FIG. 1C), thereby demonstrating that Ca²⁺ dependent inactivation of CRAG channels was reduced in crt^{-/-} cells. To confirm the mitochondrial contribution to this phenomenon, we repeated the same experiment in the presence of CCCP, a mitochondrial uncoupler. When mitochondrial calcium uptake was inhibited, the Ca²⁺ dependent inactivation of CRAC channels was comparable in both crt^{-/-} and crt^{+/+} cells (FIG. 1D).

The measurement of the mitochondrial mass by FACS analysis of noracridine orange stained cells revealed no differences between crt^{-/-} and crt^{+/+} cells (not shown). Similarly, the mitochondrial membrane potential determined by FACS analysis of cells stained with the fluorescent dye TMRM, which is retained in mitochondria in a voltage dependent manner, revealed no differences between crt^{-/-} and crt^{+/+} cells (FIG. 2B). Thus, crt^{-/-} CD4⁺ T cells have a reduced Ca²⁺ storage capacity in the ER combined with an enhanced mitochondrial Ca²⁺ buffering potential, which results in a slower Ca²⁺ dependent CRAC inactivation.

### Example 2

### Increased ATP Production in crt^{-/-} T Cells

Another important consequence of mitochondrial Ca²⁺ uptake is the stimulation of mitochondria metabolic activity with improved ATP synthesis (Jouaville et al., Proc Natl Acad Sci USA 96:13807-13812 (1999); Hajnoczky et al., Cell 82:415-424 (1995)). CCE induced by antigenic activation of crt^{-/-} CD4⁺ T cells might therefore be linked to a higher ATP production compared to crt^{+/+} cells. Indeed, following stimulation of ex vivo isolated naïve (FIG. 3A) and effector/memory (not shown) CD4⁺ T cells, we detected higher ATP levels in crt^{-/-} with respect to crt^{+/+} cells. The activation induced ATP synthesis was due to mitochondrial respiration, because it was abolished by treatment with oligomycin (FIG. 3B).

ATP production as a consequence of cytosolic calcium elevations is thought to cover the cell energy demand during cell activation. In addition, stimulation of purinergic receptors by ATP is thought to be involved in mitogenic stimulation of T cells (Baricordi et al., Blood 87:682-690 (1996)). ATP may be either stored in secretory granules or in the cytosol. To analyze the subcellular distribution of ATP in T cells, we incubated ex vivo purified CD4⁺ T cells with 4 nM quinacrine in culture medium for 30 min. This fluorescent compound binds with high affinity to nucleotides and is used to visualize ATP-containing vesicles (Belai and Burnstock, Neuroreport 11:5-8 (2000)). As shown in FIG. 3D, quinacrine staining showed a homogenous cytosolic distribution without any evidence of vesicular staining reminiscent of secretory granules. To further confirm the exclusive cytosolic localization of ATP in T cells, we fractionated T cell lysates on continuous sucrose gradient. The gradient fractions were analyzed for the presence of ATP using a bioluminescent assay (Lyman and De Vincenzo, Analytical Biochemistry 21:435-443 (1967)). In accordance with the quinacrine distribution, we found detectable amounts of ATP only in fractions corresponding to the cytosol, defined by the presence of Zap 70 in Western blot analysis. Interestingly, T cell stimulation with CD3 antibodies before fractionation resulted in increased recovery of ATP in the cytosolic fractions (FIG. 3E). These results exclude a regulated secretory release of ATP from activated T cells and suggest that ATP may be released through either connexin/pannexin hemichannels or the P2X7 purinergic receptor.

Extracellular ATP has been shown to cause shedding of L-selectin (CD62L) by activating a membrane metalloprotease. This shedding is thought to be inhibited by the P2X receptor antagonist oATP (Gu et al., Blood 92:946-951 (1998)). In vitro T cell activation of CD4 naïve T cells causes shedding of CD62L, which is progressively re-expressed following TCR triggering. In crt^{-/-} T cells, CD62L re-expression after activation was impaired with respect to wild-type cells, suggesting the presence of higher pericellular ATP concentrations. Indeed, addition of oATP to cultures resulted in enhanced and identical re-expression efficiency in both crt^{-/-} and wild-type cells (FIG. 3C). Accordingly, CD62L-negative cells are significantly increased in peripheral lymphoid organs of crt^{-/-} FLC (Porcellini et al., J Exp Med 203:461-471 (2006)).

### Example 3

### Protracted MAPK Activation in crt^{-/-} T Cells by Autocrine Activation of P2X Receptor

ATP has been suggested to participate as a costimulator in T cell mitogenic response (Baricordi et al., Blood 87:682-690 (1996)). Therefore, we have hypothesized that the hyper-responsiveness of crt^{-/-} T cells could be due to the increased production and release of ATP upon antigen encounter leading to the protracted MAP kinase activation and NFAT nuclear translocation (Porcellini et al., J Exp Med 203:461-471 (2006)). To get insight into the potential autocrine ATP-signaling in T cells, first we determined the composition of purinergic receptors expressed by DO11.10 T cell clones by RT-PCR. P2X1, 4 and 7 were found to be coexpressed together with P2Y1, 12, 13 and 14 (FIG. 4A). Stimulation with 1 mM ATP led to a calcium rise in 52% of the cells, when Ca²⁺ was present in the extracellular medium. ATP also triggered a moderate cytosolic calcium rise, due to IP3-mediated Ca²⁺ release from the ER stores when applied in the absence of extracellular Ca²⁺, thus confirming the presence of metabotrophic P2Y receptors (FIG. 4B). Using P2X subtype preferring agonists, we found that 18% of the cells were activated following stimulation with the P2X1- and 3-specific agonist αβMeATP, 35% of the cells were responsive to the P2X agoinst MeSATP, 33% responded to BzATP, a P2X7-specific agonist (FIG. 4C). 31% of the cells responded to 2MeSADP, which activates P2Y1, 12 and 13 and 36% were responsive to the P2Y14 agonist UDP-Glucose (FIG. 4D). Therefore, the P2 receptor subtypes detected by RT-PCR were also functionally competent in cultured DO11.10 T cell clones.

Given the higher activity-dependent ATP synthesis in crt^{-/-} T cells, we checked whether autocrine activation of P2 receptors might play a role in T cell hyper-responsiveness by analyzing MAPK activation, which is protracted in crt^{-/-} T cells. To this end, crt^{-/-} CD4⁺ T cells were stimulated with CD3 antibodies for 16 h in the presence of oligomycin to inhibit the Ca²⁺ stimulated mitochondrial ATP synthesis, PPADS as a nonspecific P2 receptor antagonist or oATP, which preferentially inhibits the P2X7 receptor. The prolonged activation of MAPK detected in untreated cells was almost completely abolished by the various pharmacological agents (FIG. 5B). However, the same treatments did not significantly affect the nuclear localization of NFAT1 (not shown). The decreased MAPK activation after treatment with oligomycin, PPADS or oATP was not caused by cellular damage due to toxicity, since propidium iodide (PI) staining of treated cells did not reveal any difference with respect to the untreated counterpart (not shown). These results suggest a costimulatory role for the activation induced ATP synthesis and release in T cell signaling. To further confirm this hypothesis, we followed ERK activation upon stimulation with CD3 antibodies. As shown in the first panel of FIG. 5A, ERK activation peaks at around 1.5 h after CD3 stimulation, transiently decreases at 3.5 h to return to high levels at 5.5-7.5 h. In order to distinguish between TCR dependent and independent ERK activation, TCR signaling was blocked at 30 min after CD3 activation with the tyrosine kinase inhibitor PP2, which inhibits TCR dependent Ick/fyn src-like kinase activity. As shown in the second panel of FIG. 5A, whereas PP2 treatment affected the TCR-dependent phosphorylation of ZAP-70, it did not significantly affect ERK phosphorylation at later time points, thus suggesting that ERK activation could be maintained independently of TCR signaling. However, when PP2 was added in combination with either PPADS (not shown) or oATP (FIG. 5A) a significant inhibition of ERK phosphorylation was observed. Accordingly, we found an increase in ERK activity when PP2 was combined with ARL67156, an inhibitor of ecto-ATPases, which prolongs the half-life of ATP in the extracellular medium. These results point to an important role of P2X receptor-dependent signal transduction in sustaining T cell activation.

### Example 4

### Dependence of IL-2 Expression and T Cell Proliferation on Extracellular ATP

Phosphorylation of ERK during the late phase of T cell activation has been shown to play a crucial role for IL-2 expression (Koike et al., J Biol Chem 278:15685-15692 (2003)). Therefore, we inferred that T cell stimulation in the presence of oATP might inhibit IL-2 expression. Indeed, stimulation of ex vivo isolated naïve (FIG. 6B) as well as effector/memory (not shown) CD4⁺ T cells with plate-bound CD3 and CD28 mAbs in the presence of oATP led to a significant reduction of IL-2 in the cell culture supernatant with respect to untreated cells. Analogous inhibition was observed by treatment with PPADS. In line with the hypothesized role of ATP in MAPK activation, the oATP and PPADS-dependent inhibition of IL-2 expression was rescued by contemporaneous addition of phorbol 12-myristate 13-acetate (PMA), which activates protein kinase C (PKC) (FIG. 6B). An inhibitory effect of oATP on peripheral blood T cell proliferation has been described (Baricordi et al., Blood 87:682-690 (1996)). As shown in FIG. 6A, oATP almost completely inhibited T cell proliferation measured as CFSE dilution in naive CD4 T cells stimulated with plate-bound CD3 and CD28 mAbs. This inhibition was partially rescued by the addition of IL-2 at 250 U/ml and was completely restored by the addition of PMA. We therefore hypothesize that ATP released from activated T cell is part of an autocrine loop, which plays an essential role in productive T cell activation.

NFAT nuclear translocation without concomitant MAPK activation, which can be obtained by ionomycin treatment, implements a transcriptional program leading to T cell anergy (Macian et al., Cell 109:719-731 (2002)). In spite of prominent MAPK inhibition, oATP did not affect NFAT nuclear translocation. Then, we hypothesized that activation of T cells in the presence of oATP could induce T cell anergy. In accordance with previous results showing reduced TCR signaling at early times after anergy induction, we found a reduced responsiveness in calcium imaging experiments of T cells previously stimulated in the presence of oATP. Similar results were obtained with ionomycin treated cells (FIG. 6D) (see also Heissmeyer et al., Nat Immunol 5:255-265 (2004)). The effect of oATP was not due to altered turn-over of the TCR/CD3 complex at the cell surface, as determined by FACS analysis (data not shown). To confirm that T cell stimulation in the presence of oATP could upregulate anergy related genes, we performed real-time PCR analysis of Egr2 and Egr3, two transcription factors specifically induced upon anergy induction (Safford et al., Nat Immunol 6:472-480 (2005)). FIG. 6C shows the significant upregulation of both Egr2 and Egr3 transcripts at early (2 h) and late (16 h) time points after T cell activation when oATP was added to the culture. Addition of PMA together with oATP to overcome the lack of MAPK activation robustly reduced Egr2 and Egr3 transcription. These results indicate that lack of P2X signaling upon T cell activation blunts MAPK activation and implements a transcriptional program characteristic of anergy.

### Example 5

### Effect of oATP in T Cell-Mediated Inflammation

We explored the possible use of oATP as a pharmacological agent to limit T cell-mediated inflammation. Adoptive transfer of RAG-2^{-/-} mice that express influenza hemagglutinin (HA) under control of the rat insulin promoter (INS-HA) with HA-specific transgenic TCR 6.5 (TCR-HA) CD4 cells provokes insulitis and rapid onset of diabetes. We treated mice twice daily with PBS (negative control) or oATP by intravenous and intraperitoneal injections from day 1 to 10 after reconstitution. Blood glucose at day 12 was normal in oATP-treated mice, whereas both untreated and PBS-treated mice displayed severe hyperglycemia (FIG. 7A). No relevant pathological findings were present in the pancreas from oATP-treated mice. In contrast, multifocal to coalescing inflammatory lesions replacing pancreatic islets were detected in PBS-treated animals (FIG. 7B). TCR-HA⁺cells of adoptively transferred mice were significantly reduced in the spleen (FIG. 7C) and barely detectable in the pancreas of oATP-treated animals (FIG. 7D). In addition, whereas in the pancreas of PBS-treated mice most transgenic T cells were activated and expressed CD69, in the pancreas of oATP-treated animals, CD69⁺ cells were undetectable (FIG. 7D). Ex vivo culture of splenocytes pulsed with HA 110-120 peptide and analysis of culture supernatants for IL-6, IFN-γ and TNF-α revealed significant reduction of these cytokines on a per-cell basis in cultures from oATP-treated mice compared to the PBS-treated control group (FIG. 7E).

To test oATP in a non-transgenic model of T cell-mediated inflammation, we induced inflammatory bowel disease (IBD) in T lymphopenic cd3ε^{-/-} mice by injecting naïve CD4⁺ T cells. As a healthy control we used cd3ε^{-/-} mice injected with naïve CD4⁺ T cells together with CD4⁺CD25⁺ cells comprising regulatory T cells (T_{reg}). Indeed, active suppression by regulatory lymphocytes and immunosuppressive cytokines were shown to control mucosal immunity and organ integrity. Starting at day 15 after cell transfer, the IBD group was daily injected intravenously either with PBS or oATP. Macroscopic analysis of the intestine 5 weeks after reconstitution revealed thickening of the bowel wall and unformed or absent stool in mice adoptively transferred with CD4⁺ cells and injected with PBS. This phenotype was significantly ameliorated in oATP-treated animals; in addition, spleen and mesenteric lymph nodes were normal in size (FIG. 8A). The inflammation score of oATP-treated mice was not significantly different from animals injected with CD4⁺ and T_{reg} cells (FIG. 8B). Strikingly, cellularity as well as representation of CD4⁺ effector/memory subset (CD44⁺62L⁻) and CD69⁺ cells in mesenteric lymph nodes and spleen of oATP-treated mice were undistinguishable from healthy controls (FIG. 8D, F). Moreover oATP significantly reduced the number of proinflammatory cytokine (IL-2, IFN-γ and TNF-α)-secreting cells. Interestingly, the number of IL-17 secreting cells, which were recently shown to synergize with IFN-γ producing cells in provoking severe intestinal inflammation (Kullberg et al., J Exp Med 203:2485-2494 (2006)), was analogous to the number in control mice injected with CD4⁺ and CD25⁺ T cells (FIG. 8E). Altogether, these results strongly support the view that inhibition of P2X receptors by oATP dampens T cell activation, proliferation as well as effector function, and inhibits tissue damage in T cell-dependent inflammation.

### Example 6

### Effect of PX10 Administration in T Cell-Mediated Inflammation

Our results point to a role for pannexin hemichannels in ATP release in T cells. To further test this hypothesis, we investigated whether inhibition of pannexin hemichannel assembly might inhibit T cell activation comparably to oATP. CFSE-loaded human T cells were stimulated with plate-bound anti-CD3/28 antibodies in the presence of 100 μM oATP or 200 μM of PX10 peptide. Four days later, the proliferation was measured by FACS analysis. Both PX10 peptide and oATP strongly inhibited T cell proliferation (FIG. 9A). Analogously, stimulation of mouse T cells in the presence of either oATP or the PX10 peptide strongly inhibited IL-2 secretion as detected by ELISA assay (FIG. 9B)

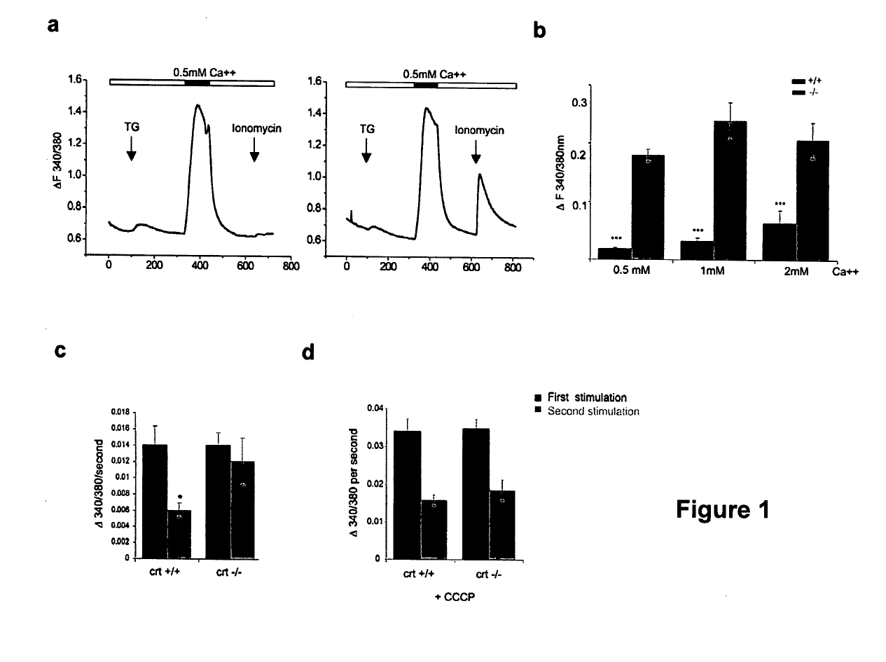

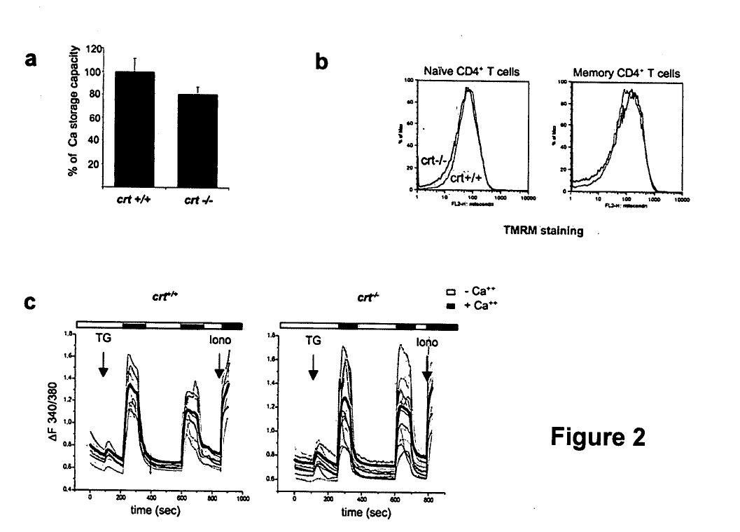

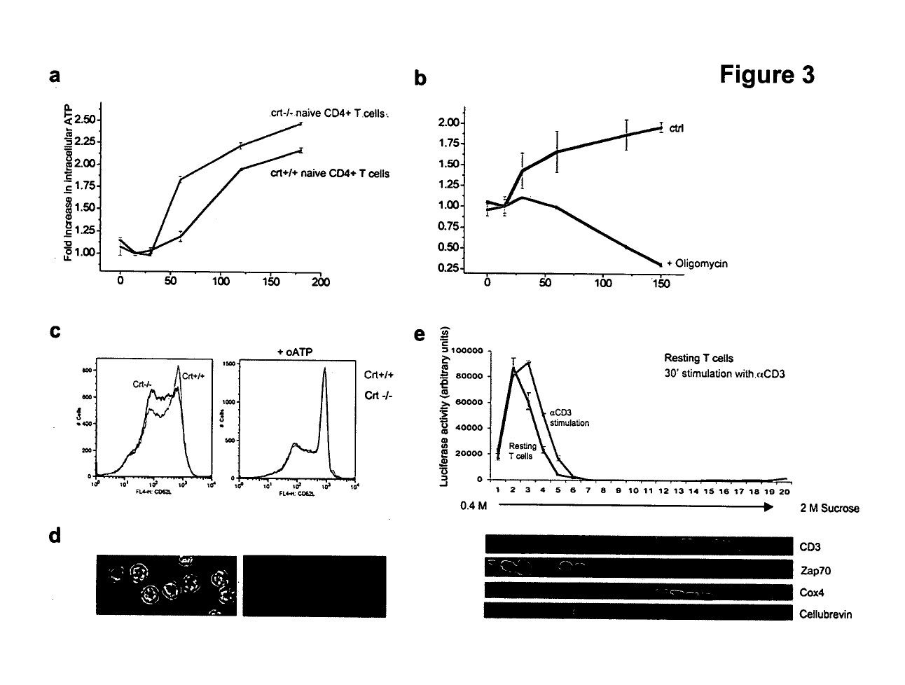

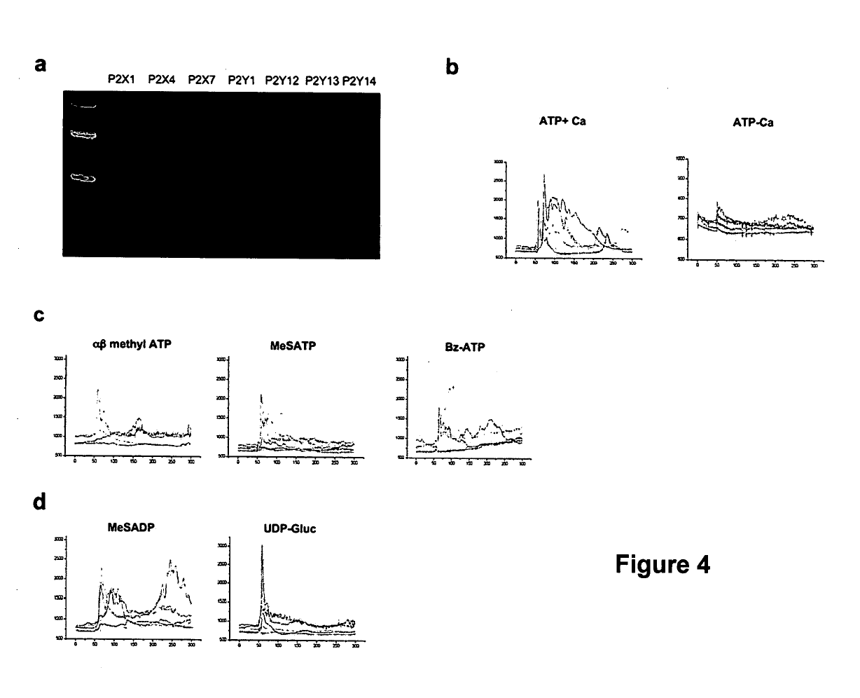

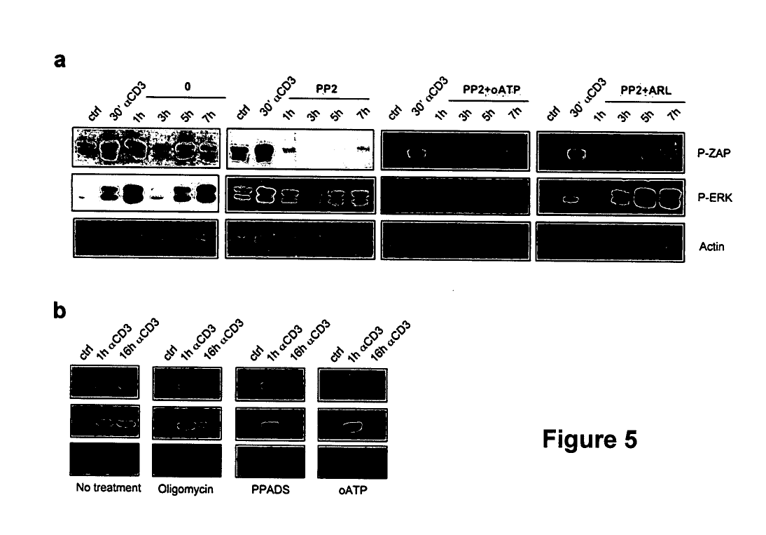

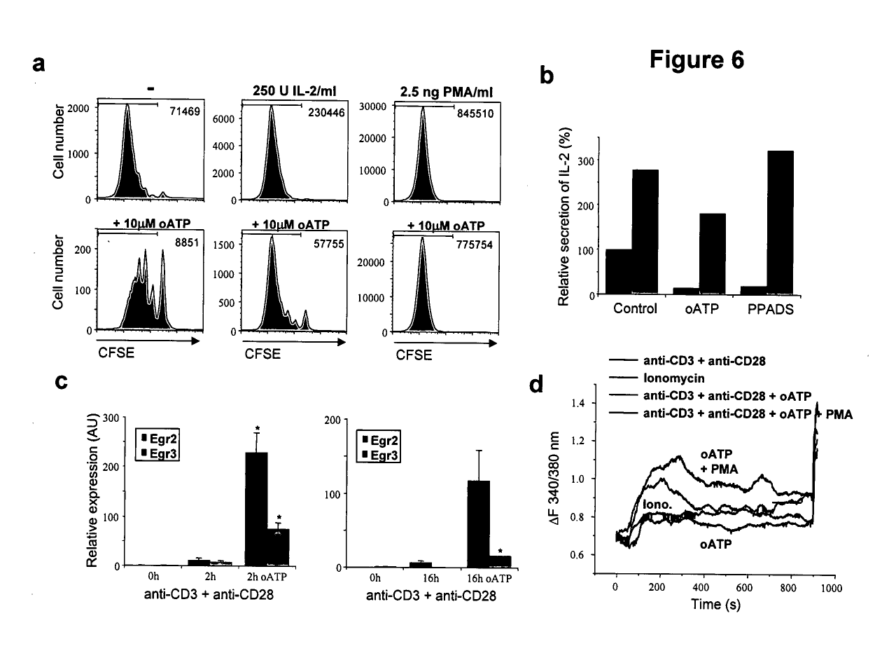

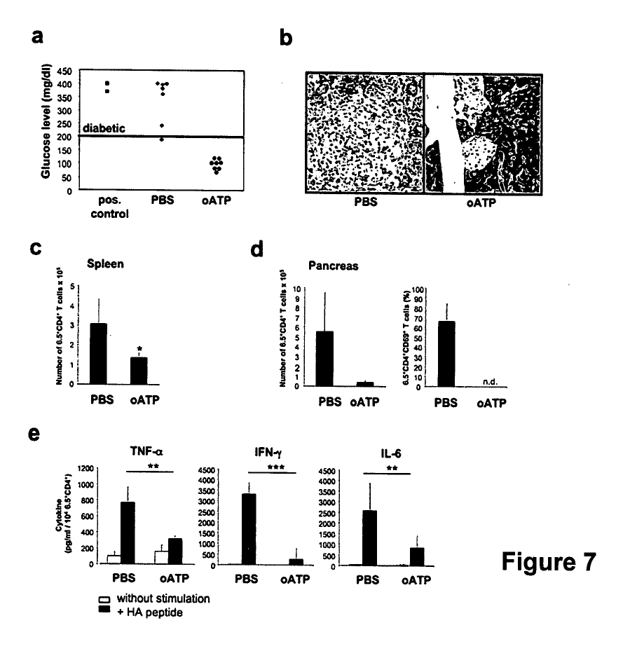

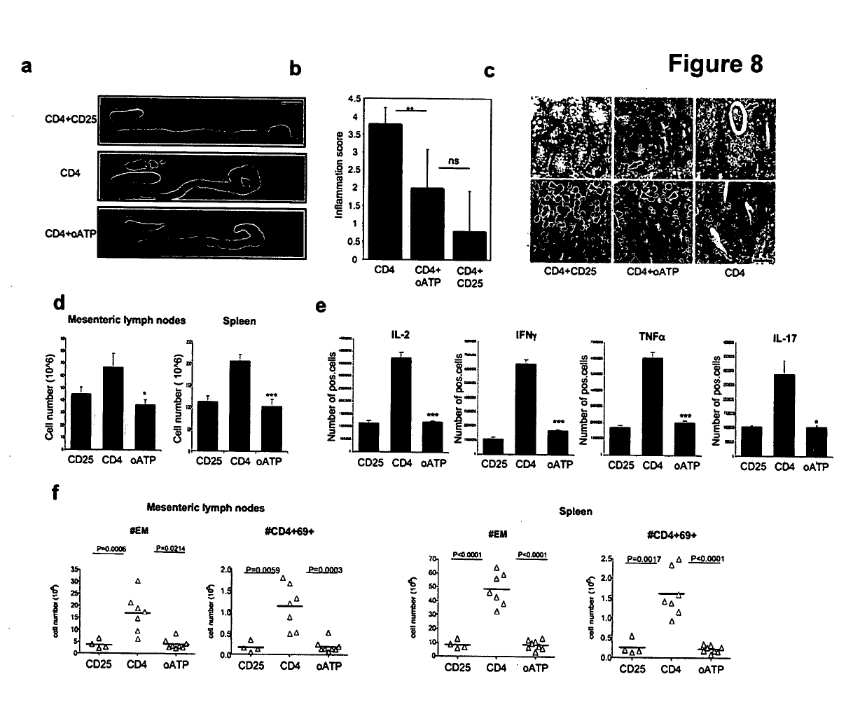

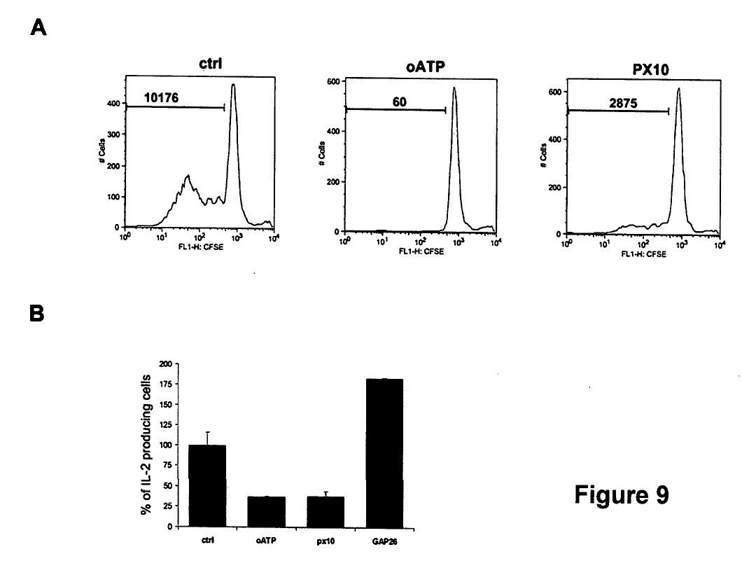

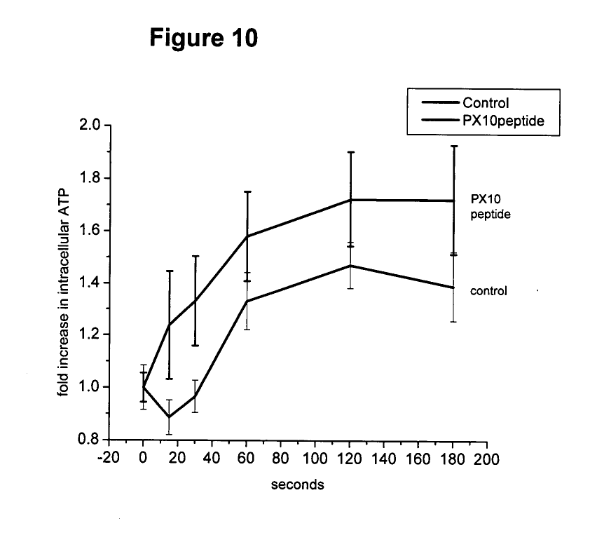

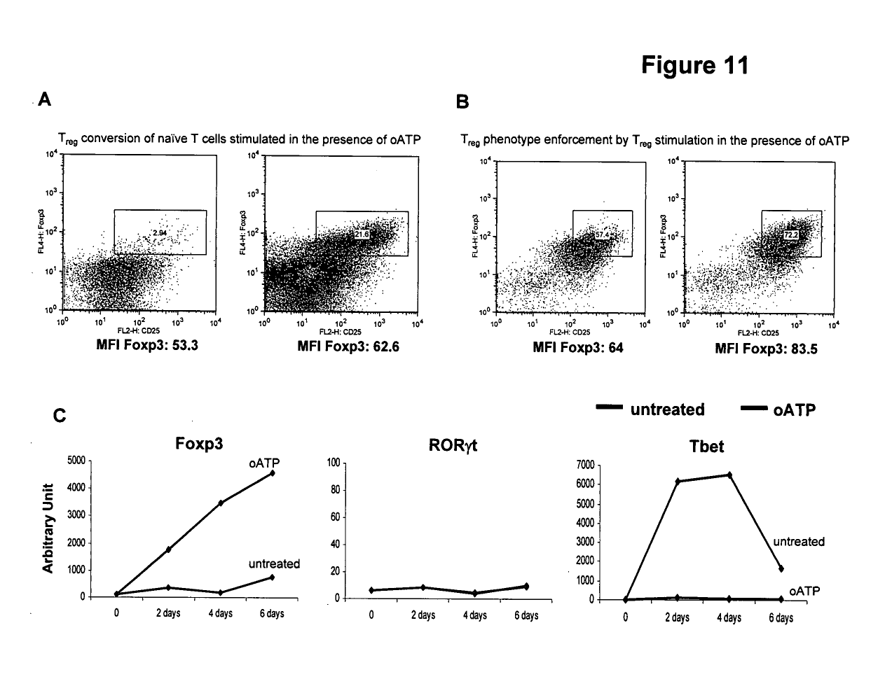

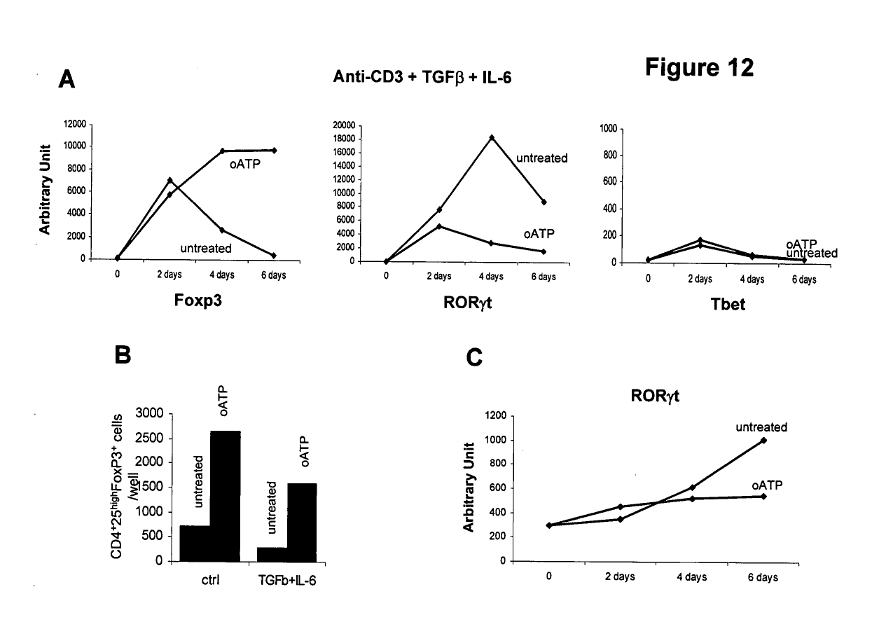

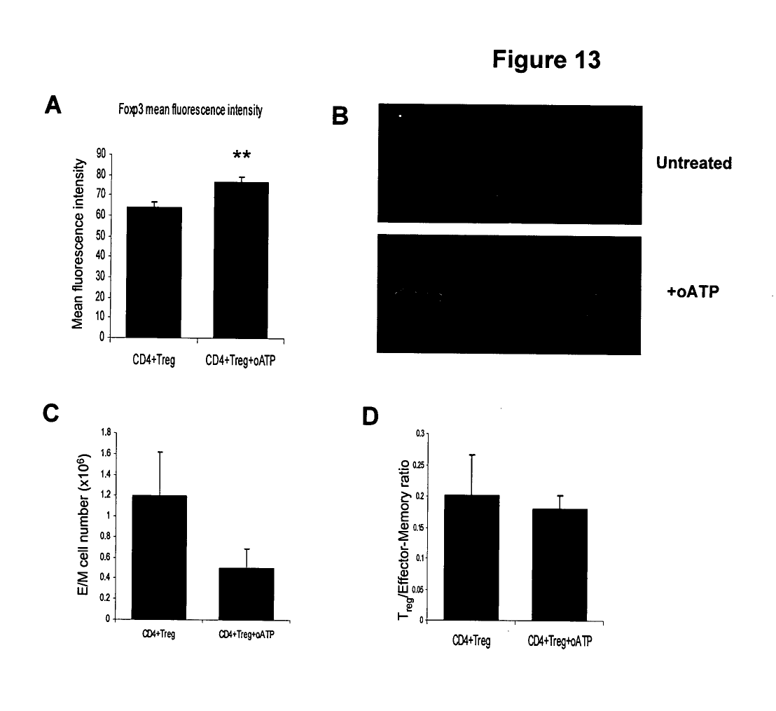

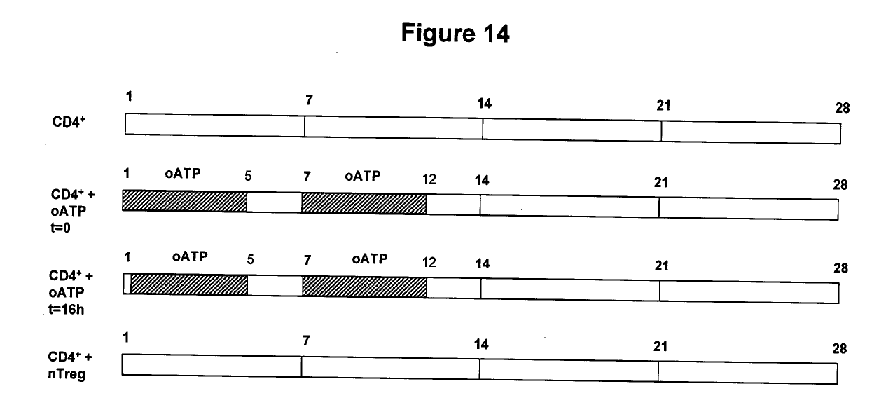

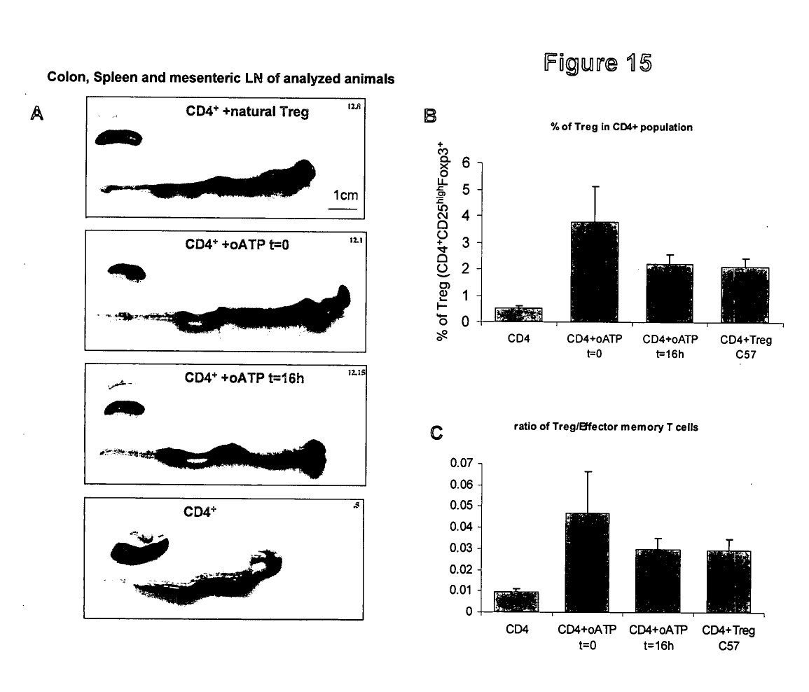

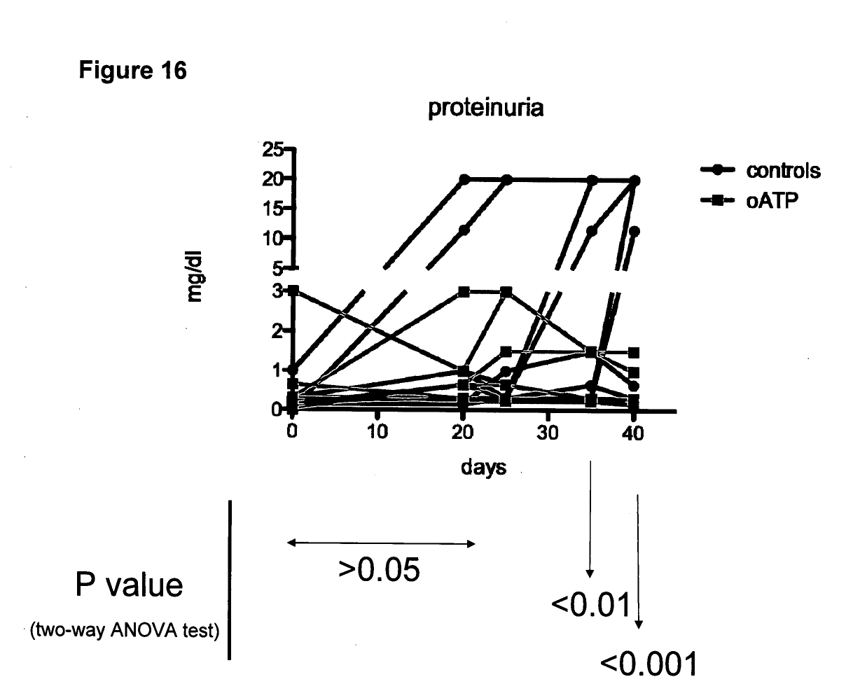

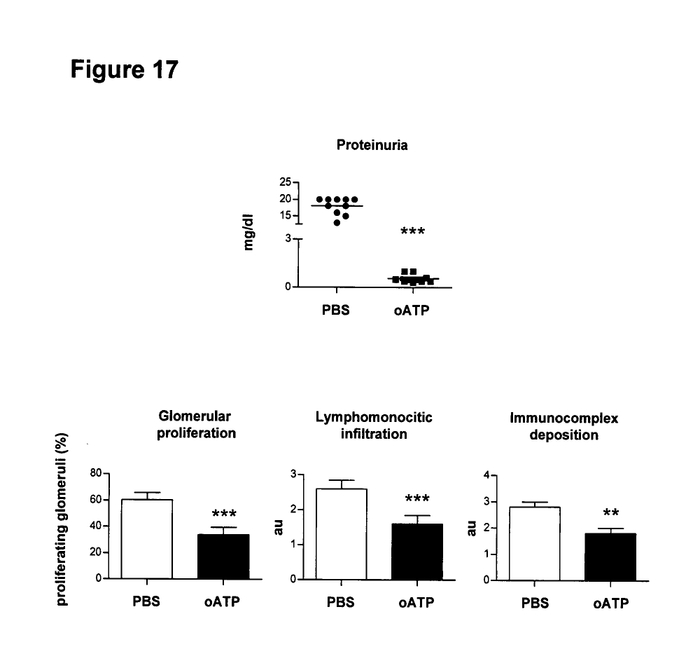

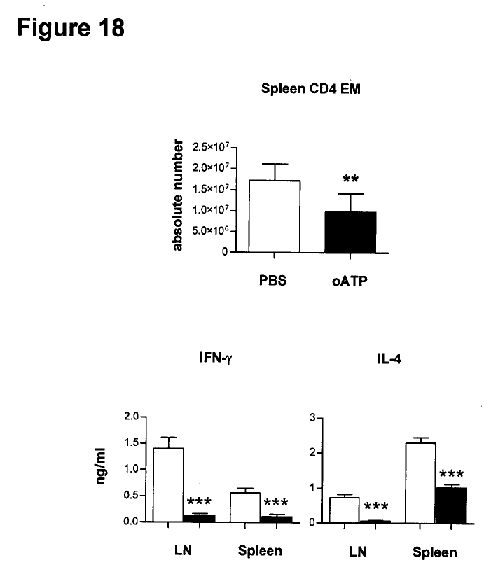

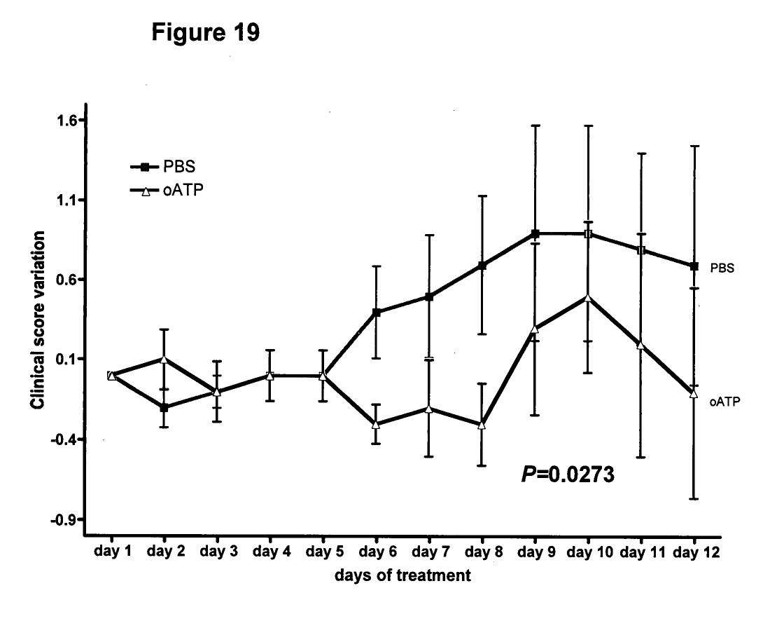

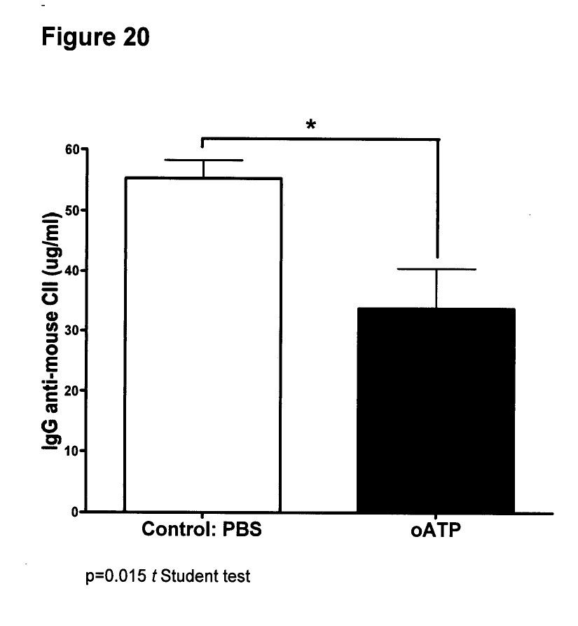

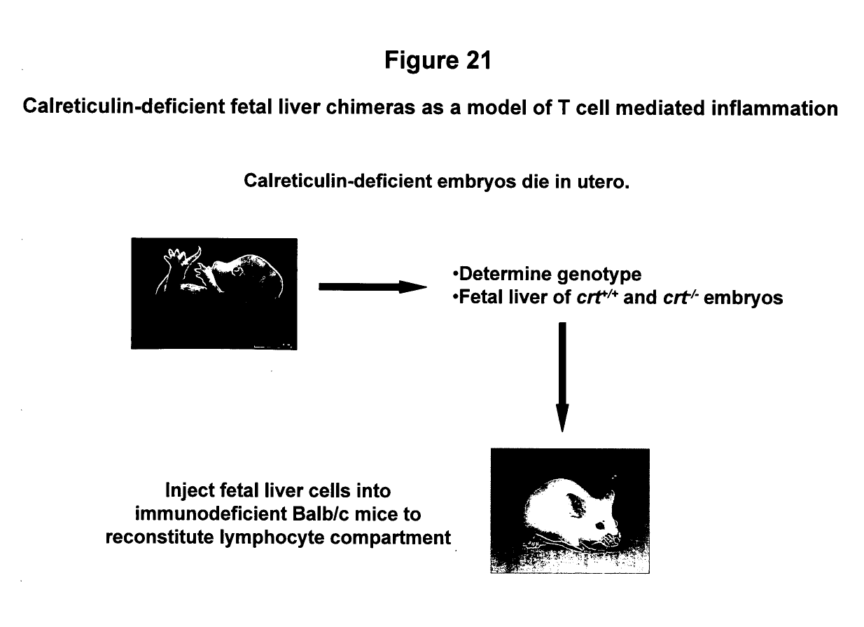

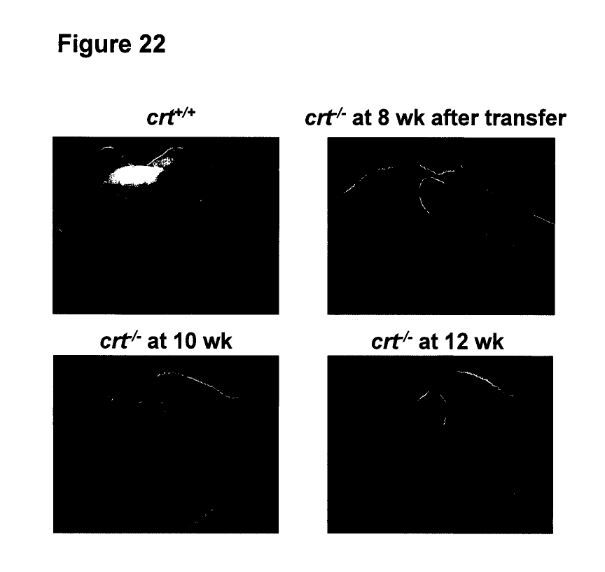

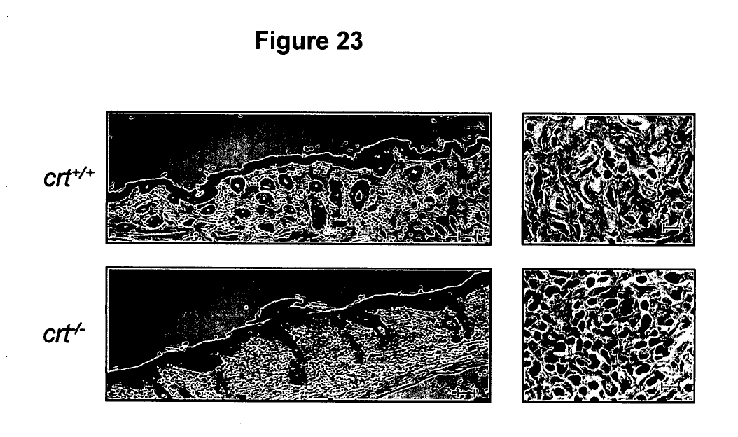

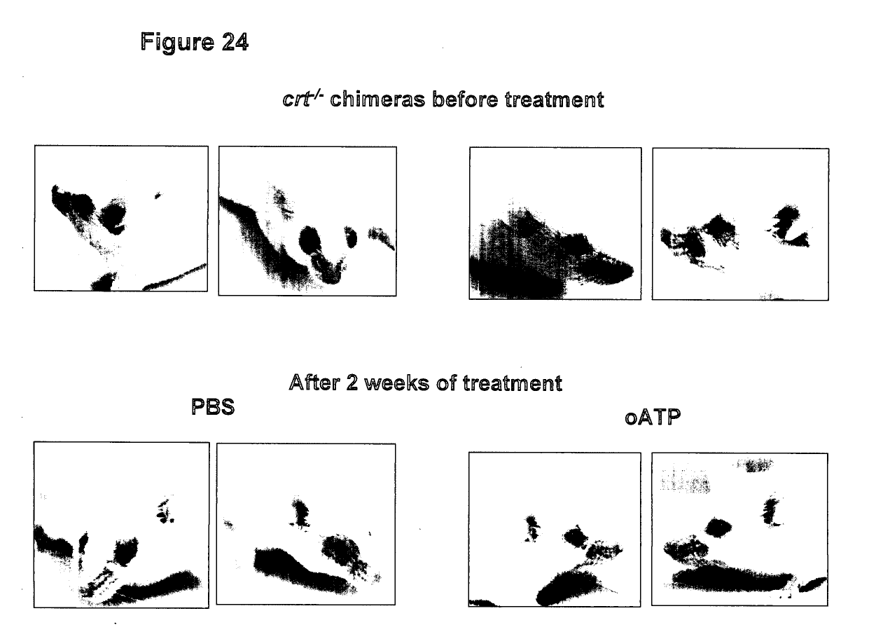

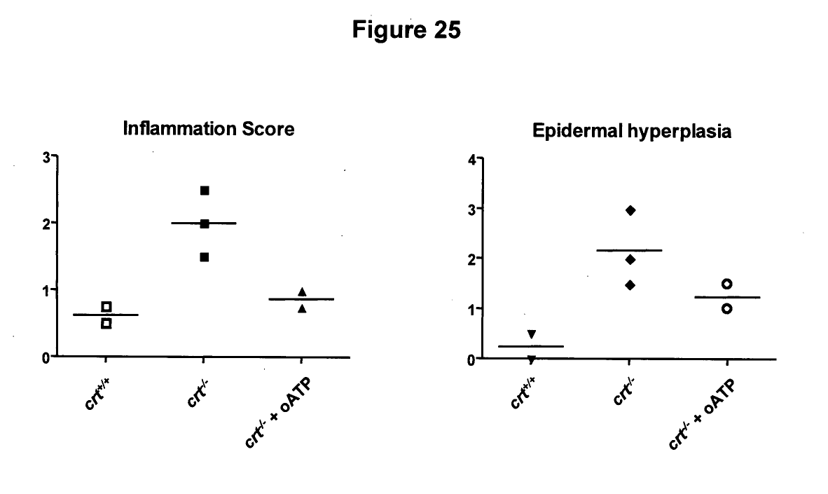

Finally, we tested whether the inhibition of pannexin-mediated ATP release might lead to a fast accumulation of ATP following T cell activation. T cell clones were preincubated with 200 μM PX10 for 30 minutes. They were then stimulated with biotinylated anti-CD3 antibodies followed by crosslinking with strepavidine. Samples were collected at indicated time points, detergent-solubilized and frozen until their use in a standard luciferase assay to determine cellular ATP content. As shown in FIG. 10, PX10 led to a rapid intracellular rise of ATP, thus indicating that pannexin hemichannels represent an important route for ATP release into the extracellular medium in T cells.

### Example 7

### Administration of oATP for Treatment of Inflammatory Bowel Disease

To treat a human for inflammatory bowel disease, oATP may be administered intravenously to reach a local concentration of 100 μM. oATP may be administered once or several times a day for weeks, months, or years, or as long as oATP treatment provides a therapeutic effect to the patient. It is expected that this will ameliorate symptoms such as thickening of the bowel wall and unformed or absent stool, as well as reducing the number of proinflammatory cytokine (IL-2, IFN-γ TNF-α and IL-17)-secreting cells.

### Example 8

### Administration of oATP for Treatment of Diabetes

To treat a human for diabetes, oATP may be administered by intravenous and/or intraperitoneal injections to reach a local concentration of 100 μM. oATP may be administered once or several times a day for weeks, months, or years, or as long as oATP treatment provides a therapeutic effect to the patient. It is expected that oATP treatment will help to normalize glycemic levels.

### Example 9

### Administration of oATP for the Suppression of Transplant Rejection

Patients with diabetes are implanted with a device comprising islet cells. Preferably, the islets are allogeneic or syngeneic. oATP is administered intranodally to lymph nodes in the vicinity of, adjacent to or surrounding the site of transplantation to reach a local concentration of 100 μM.

After transplantation, the patients' blood glucose levels are monitored daily. It is expected that regulation of blood glucose levels will improve upon implantation of the device and oATP administration. oATP may be administered once or several times a day for weeks, months, or years, or as long as oATP treatment provides a therapeutic effect to the patient.

### Example 10

### Differentiation and Expansion of Treg Cells by oATP

Sorted naïve CD4⁺CD25⁻CD44⁻CD62L⁺ T cells (10⁵) were cultured together with 2.5×10⁵ T cell depleted irradiated (50 Gy) splenocytes, in medium containing 0.5 μg/ml anti-CD3 antibody and supplemented with 100 μM oATP and 50 U/ml IL-2. After 2-6 days of culture, cells were washed and resuspended in medium containing 50 U/mI IL-2. Treg cell generation was analyzed after 7-10 days of culture by flow cytometry. Stimulation of naïve CD4⁺ T cells in the presence of oATP at 100 μM significantly enhanced the percentage of CD4⁺CD25^{high}Foxp3⁺ Treg cells (FIG. 11A). Analysis of master transcription factors for Th1 (T-bet), Th17 (RORγT) and Treg (Foxp3) lineages by quantitative RT-PCR in the first 6 days after anti-CD3 stimulation revealed the progressive upregulation of Foxp3 in the presence of oATP, as opposed to the progressive upregulation of T-bet in untreated cultures. Further, stimulation of sorted CD4⁺CD25⁺ cells comprising natural Treg cells under the same conditions induced the expansion of Treg cells with higher expression levels of Foxp3 when oATP at 100 μM was added to the culture medium (FIG. 11B).

Treatment with oATP at 100 μM inhibits Th17 differentiation and promotes Foxp3 expression. By quantitative RT-PCR, we showed that oATP at 100 μM gradually increased the expression of Foxp3 while suppressing the expression of RORγT in T cells stimulated by anti-CD3 under Th17 skewing conditions (e.g., TGFβ at 10 ng/ml and IL-6 at 20 ng/ml; see also, e.g., Bettelli et al., Nature 441:235-238 (2006)) (FIG. 12A). The absolute number of CD4⁺CD25^{high} cells expressing Foxp3 by FACS analysis was increased (FIG. 12B). Moreover, the de-differentiation of sorted CD4⁺CD25^{high} natural Treg cells to the Th17 lineage following stimulation (Koenen et al., Blood 112(6):2340-2352 (2008)) was prevented by oATP at 100 μM (FIG. 12C).

### Example 11

### Efficient Suppression of Inflammatory Bowel Disease by Treg Cells Upon Treatment with oATP

In a mouse model of IBD, where a number of Treg cells insufficient to control inflammation were adoptively transferred into the animals, daily treatment with 100 μl of 3 mM oATP administered intravenously starting at day 14 from adoptive transfer increased Foxp3 expression in Treg cells measured at day 28 (FIG. 13A). oATP treated animals showed no signs of bowel inflammation as well as no increase in spleen and mesenteric lymph nodes size (FIG. 13B), and displayed reduced counts of effector/memory T cells in mesenteric lymph nodes (FIG. 13C). The higher Foxp3 expression following oATP administration may account for the higher suppressive activity of Treg cells in this setting since the ratio of Treg/EM cells in mesenteric lymph nodes was not significantly changed by oATP treatment (FIG. 13D).

### Example 12

### Effect of oATP on Treg cell Generation in a Murine Model of Inflammatory Bowel Disease

We observed generation of new Treg cells in a murine model of inflammatory bowel disease. This model is based on adoptive transfer of sorted naïve T cells (CD4⁺CD25⁻CD44⁻CD62L⁺) in CD3e^{-/-} animals, which do not have an endogenous T cell compartment. Transferred T cells are rapidly activated by bacteria in the intestinal tract, thus leading to massive bowel inflammation within 4 weeks, if their activation/expansion is not controlled by co-transferred regulatory T cells.

Four experimental groups of CD3ε^{-/-} mice were tested for Treg generation (FIG. 14). Group (1) (negative control) was adoptively transferred with 2×10⁵ naïve CD4⁺ T cells. Group (2) was adoptively transferred with 2×10⁵ naïve CD4⁺ T cells in combination with 3 mM oATP in 100 μl. Group (3) was adoptively transferred with 2×10⁵ naïve CD4⁺ T cells and received a first treatment of 3 mM oATP in 100 μl 16 hours following adoptive transfer. Group (4) was adoptively transferred with 2×10⁵ naïve CD4⁺ T cells in combination with 10⁵ natural Treg cells (positive control). Groups 2 and 3 received daily intravenous administration of 3 mM oATP in 100 μl on days 2-5 and 8-12, with no oATP administration on days 6 and 7. Mice were analyzed on day 28.

The colons of the animals were assessed for inflammation 14 days after the last injection of oATP (FIG. 15A). Further, spleens and mesenteric lymph nodes (LN) (as draining lymph nodes) were assessed for the presence of CD4+ subpopulations. FACS analysis was performed to identify CD4⁺CD25^{high} Foxp3 regulatory T cells (FIG. 15B). Effector memory T cells were identified as CD4⁺CD44⁺CD62L⁻ or CD4⁺CD25⁺CD69⁺. In this experimental model, the ratio between Tregs and effector memory T cells in the mesenteric LN, but not in the spleen, seems to be crucial to prevent the onset of disease (FIG. 15C). Further, Treg generation from naïve T cells is most effective when oATP is present during the first activation of the naïve T cells.

### Example 13

### Use of Differentiated and/or Expanded Treg Cells to Treat Transplant Rejection or Graft-Versus-Host Disease

Prior to the transplant surgery, Treg cells are differentiated and expanded as described in Example 10, with alloactivation using the recipient cells. The resulting Treg cell population is then tested for cell viability and sterility, as well as the presence of any contaminants, before administration at and/or around the site of transplantation before and/or at the time of surgery. If necessary, subsequent doses of similarly differentiated and expanded Treg cells are administered to the subject as long as the administration is of benefit.

### Example 14

### Amelioration of Glomerulonephritis in NZB/NZW F1 Mice Upon Treatment with oATP

Systemic lupus erythematosus (SLE), or its mouse model NZB/NZW F1 (Andrews et al., J. Exp. Med. 148:1198-1215 (1978)), is a chronic inflammatory disease characterized by polyclonal B cell activation with subsequent hypergammaglobulinemia and organ injury caused by immune complex deposits. Because CD4⁺ T cells play a crucial role in the onset and propagation of the disease, we analyzed whether oATP treatment might ameliorate disease symptoms. Female NZB/NZW F1 mice at 25 weeks old (when most animals display proteinuria) received either PBS or oATP intravenously (3 mM in 100 μl, five days treatment, 2 days break) for 6 weeks. The proteinuria values were measured before the first treatment and at regular intervals after three weeks of oATP administration. The majority of PBS treated animals displayed progressively increasing proteinuria values, whereas in oATP treated mice, proteinuria levels remained similar to pre-treatment levels (FIG. 16 and FIG. 17, upper panel). Histopathological scoring of kidneys showed a significant reduction in glomerular proliferation, lymphomonocytic infiltration and immune complex deposition in oATP treated animals (FIG. 17, lower panels). Furthermore, oATP administration significantly reduced the number of effector/memory T cells in spleen and lymph nodes (FIG. 18, upper panel). Restimulation of sorted effector/memory T cells led to lower levels of IL-4 and IFNγ secretion in cells derived from oATP treated animals (18. 16, lower panels).

### Example 15

### Effect of oATP on a Murine Model of Rheumatoid Arthritis

DBA/1 mice, which are susceptible to collagen-induced arthritis, are a murine model of rheumatoid arthritis (RA) (Stuart et al., J. Clin. Invest. 69:673-683 (1982)). DBA/1 mice were immunized (day=0) by intradermally injecting at the base of the tail 0.2 ml of an emulsion composed of 200 μg bovine type II collagen in Complete Freund's Adjuvant containing 0.2 mg of Mycobacterium tuberculosis. In general, this procedure results in the appearance of signs of inflammation affecting one or more limbs, starting from approximately day 18-20.

Starting from day 18, the animals were individually graded for disease severity by means of a clinical score composed as follows:
a) Visual clinical score for the presence of inflammation in the fingers of the forepaws and hindpaws
   0=no sign of disease
   0.5=from 1 to 5 fingers/toes with signs of inflammation
   1=from 6 to 10 fingers/toes with signs of inflammation
   1.5=from 11 to 15 fingers/toes with signs of inflammation
   2=from 16 to 20 fingers/toes with signs of inflammation
b) Clinical swelling score for the presence of paw edema in the forepaws and hindpaws.Forepaws and hindpaws are measured daily for paw thickness by means of a precision caliper.
Forepaw:
   0=no sign of disease (paw thickness up to 1.29 mm)
   0.5=paw thickness between 1.30 and 1.49 mm
   1=paw thickness between 1.50 and 1.89 mm
   2=paw thickness between 1.90 and 2.20 mm
   3=paw thickness>2.20 mm
Hindpaw:
   0=no sign of disease (paw thickness up to 1.99 mm)
   0.5=paw thickness between 2.00 and 2.19 mm
   1=paw thickness between 2.20 and 2.59 mm
   2=paw thickness between 2.60 and 3.00 mm
   3=paw thickness>3.00 mm
      The sum of the two separate clinical scores (for signs of inflammation in fingers and in paws) will generate a total clinical score. The maximum swelling score per animal is therefore 14.

Mice were treated 18-20 days after immunization when they reached a clinical score of at least 1.5. Mice received oATP (3 mM in 100 μl) or control PBS intravenously in a dosage schedule of five days treatment, 2 days break, and five days treatment, starting at day 0 for a total of 12 days. The mean variation from the initial clinical score was assessed each day for oATP-treated and control groups. The mice were sacrificed at day 13. After the initial five days of treatment, the clinical score variation in the oATP-treated group remained consistently lower than the clinical score variation in the control group (FIG. 19).

A type II collagen ELISA was performed on samples from the oATP-treated and control mice, using Mouse IgG anti-Collagen Type II ELISA cat. # CIIAB96-M by MDBiosciences, Europe, division of Morwell Diagnostics, Zurich, Switzerland. oATP treatment decreased the presence of collagen-specific antibodies in treated mice compared to control mice (FIG. 20).

### Example 16

### Amelioration of Phenotype in crt^{-/-} Mice Upon Treatment with oATP

Recombinase-deficient Balb/c mice were reconstituted with hematopoietic progenitors from fetal liver of calreticulin (crt)^{-/-} and +/+E13 embryos (FIG. 21). The phenotype of the resulting crt^{-/-} fetal liver chimera (FLC), which mimics graft-versus-host disease (GVHD) (see, e.g., Porcellini et al., J Exp Med 203:461-471 (2006)), was assessed at weeks 8, 10 and 12 after transfer and compared to the phenotype of the crt^{+/+} FLC at week 12. Compared to crt^{+/+} FLC, the crt^{-/-} FLC displayed progressive worsening of alopecia, blepharitis, hunched posture and wasting syndrome (FIG. 22). Hematoxylin and eosin staining of the skin showed severe dermal granulocytic inflammatory infiltrate in the skin of crt^{-/-} FLC, as opposed to crt^{+/+} FLC, where inflammatory cells were absent (FIG. 23).

crt^{+/+} and crt^{-/-} FLCs received daily intravenous treatment with PBS or 6 mM oATP (100 μL) for two weeks. Treatment with oATP dramatically ameliorated blepharitis in crt^{-/-} mice (FIG. 24). Further, histopathological evaluation of skin biopsies executed in a blinded fashion showed that treatment with oATP resulted in histological improvement of blepharitis (both inflammation and epidermal hyperplasia) in crt^{-/-} FLCs (FIG. 25).

All publications and patent applications cited in this specification are incorporated herein by reference as if each individual publication or patent application were specifically and individually indicated to be incorporated by reference.

1-73. (canceled)

74. A method for treating a T lymphocyte-dependent immune or inflammatory condition using an agent that inhibits ATP-mediated T cell activation and which further has one or both properties of:
a) inducing T cell anergy; and
b) inhibiting the function of pannexin hemichannels.

75. The method according to claim 74, wherein said agent is oATP or a peptide comprising the amino acid sequence of SEQ ID NO: 1.

76. The method according to claim 74, wherein said T cells are IL-17-secreting T cells.

77. The method according to claim 74, wherein said T lymphocyte-dependent inflammatory condition is selected from type I diabetes, inflammatory bowel disease, multiple sclerosis, rheumatoid arthritis, transplant rejection, graft-versus-host disease, or a dermatological condition.

78. The method according to claim 74, wherein said agent is nanoencapsulated.

79. A composition comprising a P2X receptor antagonist and further comprising at least one of:
a) a T cell primary stimulator comprising one or more agents selected from a ligand that binds to the T cell receptor or a protein kinase C activator;
b) a cellular component selected from irradiated splenocytes, mobilized cell products, leukopheresis cell products, iliac crest cell products and/or vertebral bodies; and
c) a soluble mediator selected from retinoic acid, rapamycin, 5-azacytidine, trichostatin A, alphal-antitrypsin, TGF-beta, interleukin (IL)-2), CD80, 4-1BB, CD52 agonists, CD28 antibodies, lymphocyte function associated antigen-3 (LFA-3), CD2, CD40, CD80/B7-1, CD86/B7-2, OX-2, CD70 and CD82.

80. The composition according to claim 79, wherein said P2X receptor antagonist is oATP.

81. A method for promoting mammalian progenitor cell differentiation into Treg cells, comprising the step of contacting a cell capable of differentiating into a Treg cell with a composition comprising a P2X receptor antagonist.

82. A method comprising the step of contacting a Treg cell with a composition comprising a P2X receptor antagonist; wherein said composition:
a) promotes the expansion/differentiation of the Treg cell;
b) inhibits the conversion of the Treg cell to a non-Treg cell; or
c) enhances the activity of the Treg cell.

83. The method according to claim 81 or 82, wherein said composition comprising a P2X receptor antagonist further comprises at least one of:
a) a T cell primary stimulator;
b) a cellular component; and
c) a soluble mediator.

84. The method according to claim 83, wherein said composition comprises at least one of:
a) a T cell primary stimulator comprising one or more agents selected from a ligand that binds to the T cell receptor or a protein kinase C activator;
b) a cellular component selected from irradiated splenocytes, mobilized cell products, leukopheresis cell products, iliac crest cell products and/or vertebral bodies; and
c) a soluble mediator selected from retinoic acid, rapamycin, 5-azacytidine, trichostatin A, alphal-antitrypsin, TGF-beta, interleukin (IL)-2), CD80, 4-1BB, CD52 agonists, CD28 antibodies, lymphocyte function associated antigen-3 (LFA-3), CD2, CD40, CD80/B7-1, CD86/B7-2, OX-2, CD70 and CD82.

85. The method according to claim 81 or 82, wherein said P2X receptor antagonist is oATP.

86. The method according to claim 81 or 82, wherein said P2X receptor antagonist is nanoencapsulated.

87. The method according to claim 81 or 82, wherein said method is performed in vivo.

88. The method according to claim 87, wherein said method is used to treat:
a) a subject in need of Treg cells; or
b) an immune or inflammatory condition in a subject.

89. The method according to claim 88, wherein said immune or inflammatory condition is selected from:
a) transplant rejection;
b) graft-versus-host disease;
c) inflammatory bowel disease;
d) type I diabetes;
e) multiple sclerosis;
f) rheumatoid arthritis;
g) a dermatological condition selected from psoriasis, cutaneous T-cell lymphoma, cutaneous graft-versus-host disease, atopic dermatitis, allergic contact dermatitis, alopecia areata, vitiligo, drug-related eruptions, contact hypersensitivity, lupus erythematosus, pityriasis lichenoides et varioliformis, pityriasis lichenoides chronica, eczema and lichen planus; and
h) a condition associated with degranulation of mastocytes selected from asthma, allergy and anaphylactic shock.

90. The method according to claim 88, wherein said immune or inflammatory condition is a T lymphocyte-dependent inflammatory condition.


SEQUENCE LISTING (1 sequence)

SEQ ID NO: 1 - protein, 10 aa, Artificial Sequence
  Description of Artificial Sequence Synthetic peptide
WRQAAFVDSY